SEQUENCE LISTING (48 sequences)

SEQ ID NO: 1 - DNA, 3486 bp, Artificial Sequence
  Description of Artificial Sequence/Note=Synthetic Construct
ccgaagcagggcgcgcagcagcgctgagtgccccggaacgtgcgtcgcgcccccagtgtccgtcgcgtccgccgcgccccgggcggggatggggcggccagactgagcgccgcacccgccatccagacccgccggccctagccgcagtccctccagccgtggccccagcgcgcacgggcgatggcgaaggcgacgtccggtgccgcggggctgcgtctgctgttgctgctgctgctgccgctgctaggcaaagtggcattgggcctctacttctcgagggatgcttactgggagaagctgtatgtggaccaggcggccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccagtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcgtacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtactgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctagcaccgctgtcccctctgcactatccttcctctctgtgatgctttttaaaaatgtttctggtctgaacaaaaaaaaaaaaaaaaaaaa

SEQ ID NO: 2 - DNA, 3338 bp, Artificial Sequence
  Description of Artificial Sequence/Note=Synthetic Construct
gacgccaccgggcgctgacagacctatggagagtcagggtgtgcctcccgggccttatcgggccaccaagctgtggaatgaagttaccacatcttttcgagcaggaatgcctctaagaaaacacagacaacactttaaaaaatatggcaattgtttcacagcaggagaagcagtggattggctttatgacctattaagaaataatagcaattttggtcctgaagttacaaggcaacagactatccaactgttgaggaaatttcttaagaatcatgtaattgaagatatcaaagggaggtggggatcagaaaatgttgatgataacaaccagctcttcagatttcctgcaacttcgccacttaaaactctaccacgaaggtatccagaattgagaaaaaacaacatagagaacttttccaaagataaagatagcatttttaaattacgaaacttatctcgtagaactcctaaaaggcatggattacatttatctcaggaaaatggcgagaaaataaagcatgaaataatcaatgaagatcaagaaaatgcaattgataatagagaactaagccaggaagatgttgaagaagtttggagatatgttattctgatctacctgcaaaccattttaggtgtgccatccctagaagaagtcataaatccaaaacaagtaattccccaatatataatgtacaacatggccaatacaagtaaacgtggagtagttatactacaaaacaaatcagatgacctccctcactgggtattatctgccatgaagtgcctagcaaattggccaagaagcaatgatatgaataatccaacttatgttggatttgaacgagatgtattcagaacaatcgcagattattttctagatctccctgaacctctacttacttttgaatattacgaattatttgtaaacattttgggcttgctgcaacctcatttagagagggttgccatcgatgctctacagttatgttgtttgttacttcccccaccaaatcgtagaaagcttcaacttttaatgcgtatgatttcccgaatgagtcaaaatgttgatatgcccaaacttcatgatgcaatgggtacgaggtcactgatgatacataccttttctcgatgtgtgttatgctgtgctgaagaagtggatcttgatgagcttcttgctggaagattagtttctttcttaatggatcatcatcaggaaattcttcaagtaccctcttacttacagactgcagtggaaaaacatcttgactacttaaaaaagggacatattgaaaatcctggagatggactatttgctcctttgccaacttactcatactgtaagcagattagtgctcaggagtttgatgagcaaaaagtttctacctctcaagctgcaattgcagaacttttagaaaatattattaaaaacaggagtttacctctaaaggagaaaagaaaaaaactaaaacagtttcagaaggaatatcctttgatatatcagaaaagatttccaaccacggagagtgaagcagcactttttggtgacaaacctacaatcaagcaaccaatgctgattttaagaaaaccaaagttccgtagtctaagataactaactgaattaaaaattatgtaatacttgtggaactttgataaatgaagccatatctgagaatgtagctactcaaaaggaagtctgtcattaataaggtatttctaaataaacacattatgtaaggaagtgccaaaatagttatcaatgtgagactcttaggaaactaactagatctcaattgagagcacataacaatagatgataccaaatactttttgtttttaacacagctatccagtaaggctatcatgatgtgtgctaaaattttatttacttgaattttgaaaactgagctgtgttagggattaaactataattctgttcttaaaagaaaatttatctgcaaatgtgcaagttctgagatattagctaatgaattagttgtttggggttacttctttgtttctaagtataagaatgtgaagaatatttgaaaactcaatgaaataattctcagctgccaaatgttgcactcttttatatattctttttccacttttgatctatttatatatatgtatgtgtttttaaaatatgtgtatattttatcagatttggttttgccttaaatattatccccaattgcttcagtcattcatttgttcagtatatatattttgaattctagttttcataatctattagaagatggggatataaaagaagtataaggcaatcatatattcattcaaaagatatttatttagcaactgctatgtgcctttcgttgttccagatatgcagagacaatgataaataaaacatataatctcttccataaggtatttattttttaatcaagggagatacacctatcagatgtttaaaataacaacactacccactgaaatcagggcatatagaatcattcagctaaagagtgacttctatgatgatggaacaggtctctaagctagtggttttcaaactggtacacattagactcacccgaggaattttaaaacagcctatatgcccagggcctaacttacactaattaaatctgaattttggggatgttgtatagggattagtattttttttaatctaggtgattccaatattcagccaactgtgagaatcaatggcctaaatgctttttataaacatttttataagtgtcaagataatggcacattgactttattttttcattggaagaaaatgcctgccaagtataaatgactctcatcttaaaacaaggttcttcaggtttctgcttgattgacttggtacaaacttgaagcaagttgccttctaatttttactccaagattgtttcatatctattccttaagtgtaaagaaatatataatgcatggtttgtaataaaatcttaatgtttaatgactgttctcatttctcaatgtaatttcatactgtttctctataaaatgatagtattccatttaacattactgatttttattaaaaacctggacagaaaattataaattataaatatgactttatcctggctataaaattattgaaccaaaatgaattctttctaaggcatttgaatactaaaacgtttattgtttatagatatgtaaaatgtggattatgttgcaaattgagattaaaattatttggggttttgtaacaatataattttgcttttgtattatagacaaatatataaataataaaggcaggcaactttcatttgcaaaaaaaaaaaaaaaaaaaaaaaaaaaaaaaaaaaaaaaaaaaaaaaaaaaaa

SEQ ID NO: 3 - DNA, 24 bp, Artificial Sequence
  Description of Artificial Sequence/Note=Synthetic Construct
tggccgtgaagatgctgaaagaga

SEQ ID NO: 4 - DNA, 24 bp, Artificial Sequence
  Description of Artificial Sequence/Note=Synthetic Construct
tgatgacatgtgggtggttgacct

SEQ ID NO: 5 - DNA, 28 bp, Artificial Sequence
  Description of Artificial Sequence/Note=Synthetic Construct
acctgctgtcagagttcaacgtcctgaa

SEQ ID NO: 6 - DNA, 24 bp, Artificial Sequence
  Description of Artificial Sequence/Note=Synthetic Construct
ccagtacctactccctctccgtga

SEQ ID NO: 7 - DNA, 24 bp, Artificial Sequence
  Description of Artificial Sequence/Note=Synthetic Construct
tgtactggacgttgatgccactga

SEQ ID NO: 8 - DNA, 26 bp, Artificial Sequence
  Description of Artificial Sequence/Note=Synthetic Construct
tttgcccagatcgggaaagtctgtgt

SEQ ID NO: 9 - DNA, 24 bp, Artificial Sequence
  Description of Artificial Sequence/Note=Synthetic Construct
tgcaatgggtacgaggtcactgat

SEQ ID NO: 10 - DNA, 24 bp, Artificial Sequence
  Description of Artificial Sequence/Note=Synthetic Construct
ccagcaagaagctcatcaagatcc

SEQ ID NO: 11 - DNA, 28 bp, Artificial Sequence
  Description of Artificial Sequence/Note=Synthetic Construct
ctcgatgtgtgttatgctgtgctgaaga

SEQ ID NO: 12 - DNA, 22 bp, Artificial Sequence
  Description of Artificial Sequence/Note=Synthetic Construct
ggagaaggcgaatttggaaaag

SEQ ID NO: 13 - DNA, 21 bp, Artificial Sequence
  Description of Artificial Sequence/Note=Synthetic Construct
caggacgttgaactctgacag

SEQ ID NO: 14 - DNA, 24 bp, Artificial Sequence
  Description of Artificial Sequence/Note=Synthetic Construct
tggccgtgaagatgctgaaagaga

SEQ ID NO: 15 - DNA, 24 bp, Artificial Sequence
  Description of Artificial Sequence/Note=Synthetic Construct
atctgaaaggcagagcagggtaca

SEQ ID NO: 16 - DNA, 24 bp, Artificial Sequence
  Description of Artificial Sequence/Note=Synthetic Construct
tgacctgcttcaggacgttgaact

SEQ ID NO: 17 - DNA, 28 bp, Artificial Sequence
  Description of Artificial Sequence/Note=Synthetic Construct
gaaagagaacgcctccccgagtgagctt

SEQ ID NO: 18 - DNA, 26 bp, Artificial Sequence
  Description of Artificial Sequence/Note=Synthetic Construct
aagactctgcaaaagtacaatagaac

SEQ ID NO: 19 - DNA, 22 bp, Artificial Sequence
  Description of Artificial Sequence/Note=Synthetic Construct
cgatggcaaccctctctaaatg

SEQ ID NO: 20 - DNA, 24 bp, Artificial Sequence
  Description of Artificial Sequence/Note=Synthetic Construct
agcaagctttgtgtgccagtcaac

SEQ ID NO: 21 - DNA, 23 bp, Artificial Sequence
  Description of Artificial Sequence/Note=Synthetic Construct
atctccctgaacctctacttact

SEQ ID NO: 22 - DNA, 22 bp, Artificial Sequence
  Description of Artificial Sequence/Note=Synthetic Construct
aactgtagagcatcgatggcaa

SEQ ID NO: 23 - DNA, 34 bp, Artificial Sequence
  Description of Artificial Sequence/Note=Synthetic Construct
acattttgggcttgctgcaacctcatttagagag

SEQ ID NO: 24 - DNA, 24 bp, Artificial Sequence
  Description of Artificial Sequence/Note=Synthetic Construct
tgcaatgggtacgaggtcactgat

SEQ ID NO: 25 - DNA, 24 bp, Artificial Sequence
  Description of Artificial Sequence/Note=Synthetic Construct
ccagcaagaagctcatcaagatcc

SEQ ID NO: 26 - DNA, 28 bp, Artificial Sequence
  Description of Artificial Sequence/Note=Synthetic Construct
ctcgatgtgtgttatgctgtgctgaaga

SEQ ID NO: 27 - DNA, 20 bp, Artificial Sequence
  Description of Artificial Sequence/Note=Synthetic Construct
gcgtttctctcttcccactc

SEQ ID NO: 28 - DNA, 20 bp, Artificial Sequence
  Description of Artificial Sequence/Note=Synthetic Construct
gcctgaagacctggaattgg

SEQ ID NO: 29 - DNA, 20 bp, Artificial Sequence
  Description of Artificial Sequence/Note=Synthetic Construct
ggaaggtgaaggtcggagtc

SEQ ID NO: 30 - DNA, 18 bp, Artificial Sequence
  Description of Artificial Sequence/Note=Synthetic Construct
ccagccgaaggagaaggg

SEQ ID NO: 31 - DNA, 19 bp, Artificial Sequence
  Description of Artificial Sequence/Note=Synthetic Construct
taccgcattccgtccactc

SEQ ID NO: 32 - DNA, 21 bp, Artificial Sequence
  Description of Artificial Sequence/Note=Synthetic Construct
ctctccttcgtggcatctatg

SEQ ID NO: 33 - DNA, 21 bp, Artificial Sequence
  Description of Artificial Sequence/Note=Synthetic Construct
ctctccttcgtggcatctatg

SEQ ID NO: 34 - DNA, 20 bp, Artificial Sequence
  Description of Artificial Sequence/Note=Synthetic Construct
ggacatccgccataacaagg

SEQ ID NO: 35 - DNA, 20 bp, Artificial Sequence
  Description of Artificial Sequence/Note=Synthetic Construct
agacggtcatagccaaggag

SEQ ID NO: 36 - DNA, 21 bp, Artificial Sequence
  Description of Artificial Sequence/Note=Synthetic Construct
acgcaatggcttcaaggttac

SEQ ID NO: 37 - DNA, 21 bp, Artificial Sequence
  Description of Artificial Sequence/Note=Synthetic Construct
ggcatagcgaccttcaatacc

SEQ ID NO: 38 - DNA, 22 bp, Artificial Sequence
  Description of Artificial Sequence/Note=Synthetic Construct
ctgacataagtggatgcgaatc

SEQ ID NO: 39 - DNA, 22 bp, Artificial Sequence
  Description of Artificial Sequence/Note=Synthetic Construct
gctgatgatcttgaggctgttg

SEQ ID NO: 40 - DNA, 22 bp, Artificial Sequence
  Description of Artificial Sequence/Note=Synthetic Construct
agggaagtttgcgaatcagaag

SEQ ID NO: 41 - DNA, 22 bp, Artificial Sequence
  Description of Artificial Sequence/Note=Synthetic Construct
gctcctcatcaatcaccatctg

SEQ ID NO: 42 - DNA, 20 bp, Artificial Sequence
  Description of Artificial Sequence/Note=Synthetic Construct
ccaccacgaagtctcaacac

SEQ ID NO: 43 - DNA, 22 bp, Artificial Sequence
  Description of Artificial Sequence/Note=Synthetic Construct
ggtctccttgaactcaatctcc

SEQ ID NO: 44 - DNA, 21 bp, Artificial Sequence
  Description of Artificial Sequence/Note=Synthetic Construct
gcagaggagtcagacacattg

SEQ ID NO: 45 - DNA, 21 bp, Artificial Sequence
  Description of Artificial Sequence/Note=Synthetic Construct
gcagaggagtcagacacattg

SEQ ID NO: 46 - DNA, 21 bp, Artificial Sequence
  Description of Artificial Sequence/Note=Synthetic Construct
cgctggtattgtcctcttcac

SEQ ID NO: 47 - DNA, 3486 bp, Artificial Sequence; identical to SEQ ID NO: 1
  Description of Artificial Sequence/Note=Synthetic Construct

SEQ ID NO: 48 - DNA, 5842 bp, Artificial Sequence
  Description of Artificial Sequence/Note=Synthetic Construct
agtgcaatactgcccaagcccgggcggggtctctgttctctggcagaggaggtcccttggcagcgggaagcgccctctctttctctcgccgccgctccgagtctgcgccctggtgccaggcgctcagctcggcgctcccctgtgctcgcccggcgcccactcattcgcagcccggccttcgtcgccgccgcctccctgctgctcctcctcctttccccagcccgccgcggccatggcggacagcgccagcgagagcgacacggacggggcggggggcaacagcagcagctcggccgccatgcagtcgtcctgctcgtcgacctcgggcggcggcggtggcggcgggggaggcggcggcggtgggaagtcggggggcattgtcatctcgccgttccgcctggaggagctcaccaaccgcctggcctcgctgcagcaagagaacaaggtgctgaagatagagctggagacctacaaactgaagtgcaaggcactgcaggaggagaaccgcgacctgcgcaaagccagcgtgaccatccaagccagggctgagcaggaagaagaattcattagtaacactttattcaagaaaattcaggctttgcagaaggagaaagaaacccttgctgtaaattatgagaaagaagaagaattcctcactaatgagctctccagaaaattgatgcagttgcagcatgagaaagccgaactagaacagcatcttgaacaagagcaggaatttcaggtcaacaaactgatgaagaaaattaaaaaactggagaatgacaccatttctaagcaacttacattagaacagttgagacgggagaagattgaccttgaaaatacattggaacaagaacaagaagcactagttaatcgcctctggaaaaggatggataagcttgaagctgaaaagcgaatcctgcaggaaaaattagaccagcccgtctctgctccaccatcgcctagagatatctccatggagattgattctccagaaaatatgatgcgtcacatcaggtttttaaagaatgaagtggaacggctgaagaagcaactgagagctgctcagttacagcattcagagaaaatggcacagtatctggaggaggaacgtcacatgagagaagagaacttgaggctccagaggaagctgcagagggagatggagagaagagaagccctctgtcgacagctctccgagagtgagtccagcttagaaatggacgacgaaaggtattttaatgagatgtctgcacaaggattaagacctcgcactgtgtccagcccgatcccttacacaccttctccgagttcaagcaggcctatatcacctggtctatcatatgcaagtcacacggttggtttcacgccaccaacttcactgactagagctggaatgtcttattacaattccccgggtcttcacgtgcagcacatgggaacatcccatggtatcacaaggccttcaccacggagaagcaacagtcctgacaaattcaaacggcccacgccgcctccatctcccaacacacagaccccagtccagccacctccgcctccacctccgccacccatgcagcccacggtcccctcagcagccacctcgcagcctactccttcgcaacattcggcgcacccctcctcccagccttaatgcatgagcttagtctgaatttcaagttgggactcatccaatggagccgtctactcaacgccaaaggcttccttctctggcatatttggatatgacttatttgcactgaggttatctaggcttcactatccattgtgttgtaaatgtttgtcagaaatgcagccagtgttgtgggtctacaacactaaccagacgactttttccatcagtgttttacttgaatcttcatgtacgtccattccctggctggaaccttcgctgtttggtatttggtatttcagcagcagtgtgcaatttttgcttggcccagagcttcattctcctggcttttaggtttgtaaaagaaaaagggatatcttttttatatttttttccatgaatctgcagaaaattactgagctgttgttaccctcctctcattataatagtgtttaccaaacataccaataattcagcactacaattcagacctttgaaaatctggctttcagtgtagaacagaaagttagatgaatcagtgcccaagacatattttctgtttaacagaactttctacagatacattttttacaggttattttcattgtgttattgacatccatgtctctcgtaaaacagatggcccaaagtaatgaatcatgtggctgtaccttctccacataaatgggatggataattatcgtatattaagatgtgattctcttttttatccttaatgttaatctacttaacctggccccctctaacatgagtcgataaatgttgtcctactcaccggtggtttcaatggctaattagaatgtgttatttgatttctgctgcagaaggcagtgtgattgtaacaaaaacaatgcggcttccccctttcgtacttcatttgtgttctcttaaaatagagtttgaacaaatattttaaaggtgcaaaataccattagaaaatactatttgaaatggacattatcgcattatcttggcataatggccagaaaatattgtattgcttggcagaaaagaaaataaggtctaaaggaaagtagcacattagcattgatggctgttcatttcacccagtataagcaagtgcagtgtacaaagaagtatattctgaatacattatttccattcatttagcacaaataaatcatttggtttcactttgcagtggaacactgagtcactcttttcttaacacgtgcaacatcttaatttttgtttttcagcagttgctgttttgtactttggtagtaaagtgatttttaccacctgtgtttgcatatttatatatgctgtggatgaaaataacttactagagaatgtatattttatgacaagaatgtgtatctgttggatataatcagagaactgaaaagtaatttatcagtaatttttaagagtccatgttttgtgacaaccatctctaatagccaactctttattaaacacactcctaaaaataaggaaccatgacattgtagatatttaatattgtacagtatagaaacctccatttttgccttcgaatgcatatttaagagttaacagaatgaaaaaaaaaagtcttgttggataatagtgtttgactagcgttttaagaacttgagagtaaaagcaacaataagattttttcacctcttcctgcttccacccccaaactgagaacatcactcaattgtttggaagaaactgtaggtctatataaattttatttataatgtatgtgtaatatacataatcataatacagttctcagatgcagggaagaagtttggcatttaatcattgaggctttaggtttttgatgtgatcagactgggccatgtcaaacccggaattttcaccaacagttcactcaccctcctggtacattgccattccaaggaattctgagagtaggcaaacaaattttgccttcatggtacagttctcagtttttcttataggagaaatatggtatatgtttataagaatcttttatgagattatagatttcaatgctgtggatagtgtcttgcacccaaacaagaaagtccataatggaatgatcttccctcagcttcctatcgatttagttacctcttgaaagcacaaaaattaaaacattgccatatgttgaatttttaaaaagcacttggagtgagcgaacatttcctgataaatgccttttagagataggttcttgatattcagacatctgcagaaatgttctggttcccaaagtcatttcacttcgaaataaaacacagctccttcaaacagcactttttccacataaatctagttgcctctccctgtggacattcagaactgatagaacaaacactactcttttgaatttgatggttcgtgtcctttaaagtgtttgaggacctatgcagagcctgtaacacttgggtagtacctgctaggacaatttcttggcaattgtcttactactagggatcagtaagatttagattctgagcccataatggcaacagccccctcacctatgggaagctgacttccctcagtcgggcacttctcatgggggctgaacatggttcctgccattctgttacccactctcccaggtgagccctggattggctcccagaaggcctttgtaaaatcagtagccgtcctgcaggcaggtgggagcaacaggggcttcagtagcttcattttcctgtcttgcagacagagacccttggctaccactgtgctgctaataggataagtactctgttgccagattaccatgccttttatacaaaaccaaattaacttacctaatacctgacacctctttgggctctgaactgctttctctcatcaagcatgctagcactctagacagaattctagaaatttggcagatagtggaagcctttaattgaacttactccttcgttgactgaaaggagttttaaattctgagctcctgagatactgactagcaaccatggaatgaatgtgtgaccagaaagtggctttgacaccaagtgctactgtccctttgtaattggcttctaacagaattcaaccagaaataattgataatgtgaatttttgttaattgttcacttgtaggaaaatagaacatgtatcaccctttgttaggtagacatgaacttttcctgcacaaagccttgcttttagagaatgcccaataaggcaagaaaaagcatagtaacttgtgctttgagagctcaatatttgtatcttatcagtacagaagaaatatttctgtgtaacttgatcttctgtctagtacttgtcttataggtaaccaacactgaaaactttgtagtgatgactaccaaagaaatacatagtaaaacaaccttttatttccaaattgttaaagagccagccattgatgctgctacatgagttccatgctcaagagccattgtaagagattaaggggtttctaggtttttggtgattttttgtttgtttttttctttgttttttagggtttttttttcttctttaattttttgattaaaacatacacacagctgttagcataaagtcgtggggggcattttctggaatgctcagcagttctgattaactgccaagcccaggttgcctctcatgaggcaactgaaaaaatcctgtgtcttgatagcatgggtgctgtgtgtgtgcatgtgtgtgtctgcattcatgccttaactcgggttactgcacaactttagttcttgacttagtctgcaccgtcatctagattgtattgtacatctcggtctgaacttcatcctggcaaaaacaaagttgcaggcacaacagtttaagaatgcattcctccagaagagtatctggtcaggttgacccctgagccttctttggacttgatttggaacttagcctggaaagcgaaagtggactgtccaacagaaagatgtcaacaaggaaaagaggagagccaagcgctagcatgccttttgcctctgcatatctgtgcacactgtatgttgttcatgatagcttgtctacaacttgactaggttggagttctggtaatagtggcaatcttgacattcttggtcagagtttagagagatgtaagactttcaattaatgtcttatttactcctttatgttgattagtctttgatacatgtgctgaatcagaaacctaaataaagataattttttaaaatgtacctcttgagccttaaaaaaaaaaaaaaaaaa

(19) United States
(12) Patent Application Publication
(10) Pub. No.: US 2015/0232940 A1
(43) Pub. Date: Aug. 20, 2015

(54) METHODS AND COMPOSITIONS RELATING TO DIAGNOSING AND TREATING RECEPTOR TYROSINE KINASE RELATED CANCERS

(71) Applicant: INSIGHT GENETICS, INC., Nashville, TN (US)

(72) Inventors: David Hout, Nashville, TN (US); John Handshoe, Bethpage, TN (US)

(73) Assignee: INSIGHT GENETICS, INC, Nashville, TN (US)

(21) Appl. No.: 14/383,735

(22) PCT Filed: Mar. 8, 2013
(86) PCT No.: PCT/US2013/029969

§ 371 Date: Sep. 8, 2014

Related U.S. Application Data
(60) Provisional application No. 61/609,069, filed on Mar. 9, 2012.

(51) Int. Cl. C12Q 1/68

(52) U.S. Cl. CPC C12Q 1/6886; C12Q 2600/158; C12Q 2600/136

(57) ABSTRACT

Disclosed are methods and compositions for detecting the presence of a cancer in a subject and assessing the efficacy of treatments for the same. The disclosed method use reverse transcription polymerase chain reaction (RT-PCR), real time polymerase chain reaction, and multiplex polymerase chain reaction techniques to detect fusions, over-expression, truncation, and nucleic acid variation of RET and DEPDC1 in cancers.

### BACKGROUND

Lung cancer leads the pact as the main cause of cancer mortality worldwide whereas ˜80% of lung cancer cases are non-small cell lung cancer (NSCLC) in type and >50% of NSCLC are adenocarcinoma in histology. Molecular drivers of adenocarcinoma NSCLC have been widely restricted to three mutually exclusive oncogenes, EGFR, KRAS and ALK (collectively between 30-60% of all NSCLC cases) leaving the remaining 40% placed into a triple negative NSCLC (EGFR, KRAS and ALK negative) category with no know oncogenic drivers. Investigations to identify specific genetic drivers of triple negative NSCLC are therefore pivotal and have recently received growing attention; various groups have in fact utilized large scale genome-wide expression profiling to investigate the biomarker profile of this NSCLC subtype. The complete absence of therapeutic targets for triple-negative adenocarcinoma NSCLC patients undeniably supports the need for identification of upregulated and mutated genes within this poor prognostic cohort.

### BRIEF SUMMARY

The methods and compositions disclosed herein relate to the field of detection or diagnosis of the presence of a disease or condition such as cancer; assessing the susceptibility or risk for a disease or condition such as cancer; the monitoring disease progression for a disease such as cancer; and the determination of susceptibility or resistance to therapeutic treatment of a disease such as cancer, wherein the disease or condition is a cancer associated with a nucleic acid variation, over-expression, truncation, or gene fusion of the DEPDC1 and/or the RET gene. It is understood and herein contemplated that the methods disclosed herein allow for rapid and sensitive detection of rare truncations and aberrant over-expression of wild-type genes.

In accordance with the purpose(s) of this invention, as embodied and broadly described herein, this invention, in one aspect, relates to methods of detecting the presence of a cancer by detecting a nucleotide variation within a nucleic acid of interest comprising conducting reverse transcription polymerase chain reaction (RT-PCR), real-time PCR, real-time RT-PCR or fluorescence in-situ hybridization on mRNA extracted from a tissue sample from a subject with a cancer; wherein the presence of amplification product or an increase in amplification product relative to a control indicates the presence of a fusion, nucleotide variation, truncation, or excessive expression, thereby detecting the presence of a cancer. In particular, the invention, in one aspect, relates to methods of detecting the presence of a cancer by detecting the presence of one or more RET and/or DEPDC1 related fusions, and/or the upregulated expression of wild-type RET and/or DEPDC1 as may occur in certain cancers.

In another aspect, disclosed herein are kits for diagnosing an RET or DEPDC1 related cancer comprising (a) a first primer labeled with a first detection reagent, wherein said first primer is a reverse primer, wherein said reverse primer is one or more polynucleotide(s) that hybridizes to a first polynucleotide encoding the amino acid sequence of SEQ ID NO 1 or SEQ ID NO 2 or the complements thereof; and (b) at least one second primer, wherein said second primer is a forward primer, wherein said forward primer is one or more polynucleotide(s) that hybridizes to a second polynucleotide encoding wild-type RET, wild-type DEPDC1, a RET fusion partner, or a kinase domain of RET.

Additional advantages of the disclosed methods and compositions will be set forth in part in the description which follows, and in part will be understood from the description, or may be learned by practice of the disclosed method and compositions. The advantages of the disclosed methods and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows a Schematic representation of receptor tyrosine kinases (RTKs) that are involved in oncogenesis due to the generation of fusion kinases. The drawings illustrate the normal, membrane-spanning RTKs. The proteins shown in red underneath each RTK form constitutively active, oncogenic fusions with the kinase.

FIG. 2 shows underlining design for RET and DEPDC1 screens. FIG. 2A shows the RET screen and primer positions for measuring over-expression and fusion detection. FIG. 2B shows primer positions used to measure DEPDC1 expression levels.

FIG. 3 shows the target design in the RET transcript. The region 3′ of known fusion breaks and the kinase domain of RET was targeted for assay design while also avoiding known inhibitor resistance hot spots (abbreviated list annotated above). Exons: green arrows; Regions for primer design: red arrows; transmembrane domain: blue arrow; kinase domain: brown arrow.

FIG. 4 shows target Amplification of the Insight RET Screen. Products from the Insight RET Screen and control ECD reaction were subject to gel electrophoresis after amplification of either full-length RET (NB-39nu) or RET fusion (TPC-1).

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a larger amount of a composition or compound, such as an amplification product relative to a control. Thus, for example, an increase in the amount in amplification products can include but is not limited to a 10%, 20%, 30%, 40%, 50%, 60%, 70%, 80%, 90%, or 100% increase. It is further contemplated herein that the detection an increase in expression or abundance of a DNA, mRNA, or protein relative to a control necessarily includes detection of the presence of the DNA, mRNA, or protein in situations where the DNA, mRNA, or protein is not present in the control.

“Obtaining a tissue sample” or “obtain a tissue sample” means to collect a sample of tissue from a subject or measure a tissue in a subject. It is understood and herein contemplated that tissue samples can be obtained by any means known in the art including invasive and non-invasive techniques. It is also understood that methods of measurement can be direct or indirect. Examples of methods of obtaining or measuring a tissue sample can include but are not limited to tissue biopsy, tissue lavage, aspiration, tissue swab, spinal tap, magnetic resonance imaging (MRI), Computed Tomography (CT) scan, Positron Emission Tomography (PET) scan, and X-ray (with and without contrast media). It is further understood that once a tissue sample is obtained, nucleic acid (e.g., DNA, RNA, or cDNA) can be isolated from the tissue sample.

The sensitive detection of a mutation at a known site in DNA is readily done with existing technologies. Allele specific primers can be designed to target a mutation at a known location such that its signal can be preferentially amplified over wild-type DNA. Unfortunately, this is not possible with unknown mutations that may occur at any position (base) in the target sequence.

It is understood and herein contemplated, that the tissue sample can come from any tissue in a body. Thus, as used herein, “tissue” refers to blood, neural tissue (e.g., brain tissue or spinal cord tissue), lymphatic tissue, hepatic tissue, splenic tissue, pulmonary tissue, cardiac tissue, gastric tissue, intestinal tissue, pancreatic tissue, tissue from the thyroid gland, salivary glands, joints, and the skin. It is understood that a tissue sample can comprise as little as a single cell or fraction from the target tissue. for example, the tissue sample can be Peripheral Blood Mononuclear Cells, B cells, T cells, Macrophage, Erythrocyte, Platelet or other blood cell. Similarly, the cell could be an epithelial cell, hypatocyte, neuron, or other cell.

Methods of Detecting a RET and/or DEPDC1-Related Cancer

The disclosed methods in one aspect related to methods of detection or diagnosis of the presence of a disease or condition such as a RET or DEPDC1 related cancer (such as, for example, non-small cell lung carcinoma, medullary thyroid carcinoma, papillary renal cell carcinoma, and hepatocellular carcinoma), assessing the susceptibility or risk for a disease or condition such as a RET or DEPDC1 related cancer (such as, for example, non-small cell lung carcinoma, medullary thyroid carcinoma, papillary renal cell carcinoma, and hepatocellular carcinoma), the monitoring of the progression of a disease or condition such as a RET or DEPDC1 related cancer (such as, for example, non-small cell lung carcinoma, medullary thyroid carcinoma, papillary renal cell carcinoma, and hepatocellular carcinoma), and the determination of susceptibility or resistance to therapeutic treatment for a disease or condition such as a RET or DEPDC1 related cancer (such as, for example, non-small cell lung carcinoma, medullary thyroid carcinoma, papillary renal cell carcinoma, and hepatocellular carcinoma) in a subject.

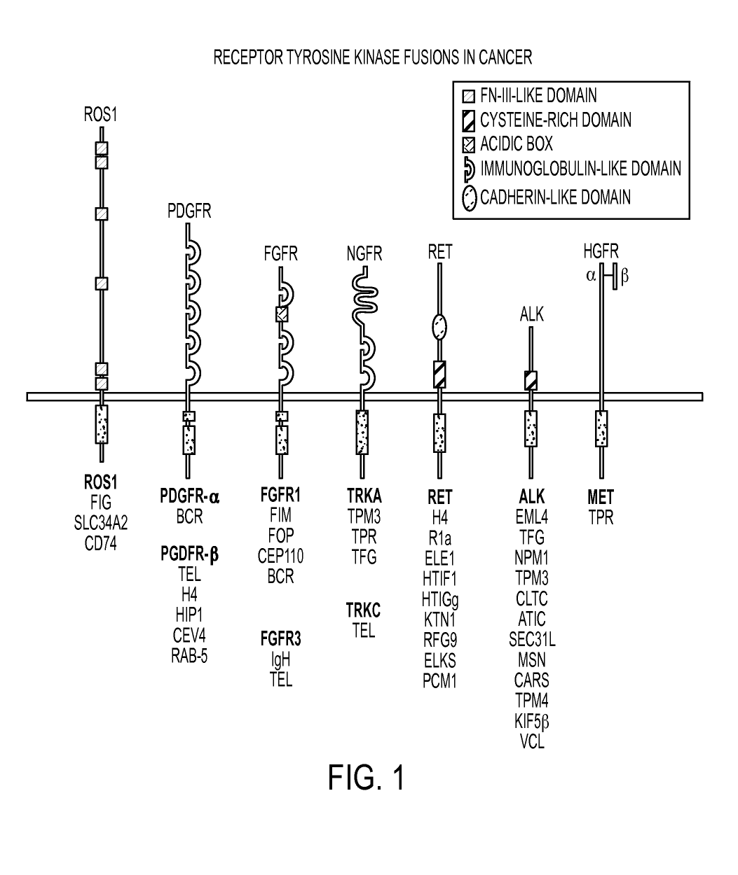

Receptor tyrosine kinases (RTKs) are important regulators of signal transduction pathways that play crucial roles in normal development by controlling cellular proliferation, differentiation, migration, and other cellular functions. Perturbations in RTK signaling through various genetic alterations can result in deregulated kinase activity and ensuing malignant transformation. One such common oncogenic mutational mechanism involves truncation of the kinase and its fusion to a heterologous N-terminal activating fusion partner (FIG. 1). The resulting chimeric protein is hyper-activated through dimerization of the kinase domains which trigger downstream signaling pathways associated with cell proliferation and survival. Oncogenic fusion of RTKs has been shown to drive a number of neoplasms in lung cancer, including fusions of the ALK and ROS1 genes

Thus, in one aspect the cancers involved in the disclosed methods are receptor tyrosine kinase related cancers such as, for example, a non-small cell lung carcinoma negative for expression of EGFR, KRAS and ALK. The disclosed methods comprise detecting the presence or measuring the expression level of nucleic acid (e.g., DNA, cDNA, RNA, and/or mRNA) in a tissue sample from the subject; wherein an increase in the amount of amplification product relative to a control indicates the presence of a cancer, (such as, for example a non-small cell lung carcinoma, medullary thyroid carcinoma, papillary renal cell carcinoma, and hepatocellular carcinoma) and wherein the cancer is associated with a nucleic acid variation, over-expression, truncation, or gene fusion resulting of a RET or DEPDC1.

Non-Small Cell Lung Carcinoma.

Non-small cell lung carcinoma is the most common type of lung cancer that includes adenocarcinomas, squamous cell carcinomas and large cell carcinomas. Among the adenocarcinomas of NSCLC, the majority result from the oncogenes Epidermal Growth Factor Receptor (EGFR), V-Ki-ras2 Kirsten rat sarcoma viral oncogene homolog (KRAS), and Anaplastic Lymphoma Kinase (ALK) including cancers which involve the formation of a chimeric protein between an oncogene such as ALK and a fusion partner. Albeit to a lesser extent adenocarcinomas can also relate to other oncogenes. This is particularly important as such EGFR, KRAS, and ALK negative cancers (a.k.a. triple negative cancers) account for 40% of NCSLCs. Among the oncogenes responsible for triple negative adenocarcinomas are RET and DEPDC1.

### RET

Receptor tyrosine kinases such as RET are cell-surface molecules that transduce signals for cell growth and differentiation. Dysregulation of RET signaling is one of the most common molecular defects associated with various malignancies. The RTK encoded by the c-ret proto-oncogene is rearranged and constitutively activated in a large proportion of thyroid papillary carcinomas. RET (which stands for REarranged during Transfection) is normally expressed in the developing central and peripheral nervous systems (sensory, autonomic and enteric ganglia) and the excretory system (Wolffian duct and ureteric bud epithelium) of mice and is a major oncogenic driver in non-small cell lung carcinoma and mudullary thyroid carcinoma. RET can be a receptor for a factor involved in the proliferation, migration, differentiation or survival of a variety of neuronal cell lineages, as well as in inductive interactions during organogenesis of the kidney. Furthermore, c-Ret expression was found in the acinar cells of the salivary gland, the epithelial cells of the thymus and the follicular dendritic cells of the spleen and lymph node in infant and adult rats. Taken together, RET is predominantly restricted to prenatal expression in both the nervous system and kidney with adult expression highly localized to distinct cell types of the lymphatic system. No expression of RET in adult lungs has been reported indicating monitoring abnormal expression of RET in lung tissue is feasible and constitutive expression of Ret is only marginal.

The expression of the RET kinase domain driven under the control of a promoter present in the Kinesin-1 heavy chain has been recently observed in a pericentric inversion fusion, KIF5B-RET, expressed exclusively in the triple negative NSCLC phenotype. The most frequently observed fusion to the RET kinase domain is the KIF5B gene (62%) with a smaller fraction fused with coiled coil domain containing 6 (CCDC6) gene (16%) and the nuclear receptor coactivator 4 (NCOA4) gene (8%). Massively paralleled whole-genome and transcriptome sequencing was used to reveal the presence of KIF5B-RET in a triple negative adenocarcinoma patient and also in a second double negative (EGFR and EML4-ALK negative) patient allowing extrapolated and estimated frequency rates of close to 6% in lung adenocarcinoma to be proposed. Support of these frequencies were further provided from the Cancer Genome Atlas (TCGA) which indicated RET over-expression is indeed observed at levels approaching 10% in lung adenocarcinoma. An additional RET fusion, CCDC6-RET, previously restricted to thyroid cancer was also identified albeit at a much lower prevalence and like KIF5B-RET exclusively to tumors of adenocarcinomas histology.

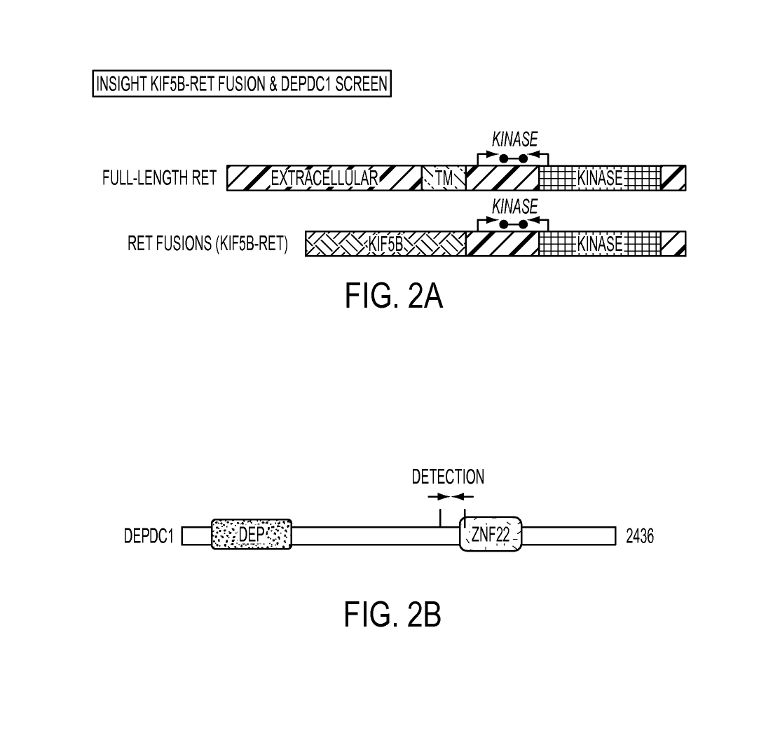

Structurally the KIF5B-RET consists of three functional domains including a kinesin motor domain, coiled-coil domain and kinase domain, the latter two of which are responsible for dimerization and subsequent activation of the kinase domain by autophosphorylation FIG. 2A. Activation of the kinase domain drives the cell into a complex cancer pathway. Comprehensive RET diagnostics are therefore pivotal to identify the full length as well as fusion forms present in adenocarcinoma patients.

In one aspect, the methods disclosed herein can be accomplished through quantitative PCR assays, semi-quantitative PCR assays, or non-quantitative PCR assays. the methods can detect the presence or the overexpression of RET for the diagnosing a RET related cancer (such as, for example, a cancer occurring involving RET overexpression or a RET fusion). Thus, disclosed herein, in one aspect, are methods of detecting the presence of (i.e., diagnosing the presence) a RET overexpression or a RET fusion in a subject with a cancer comprising detecting the presence of or measuring the amount of nucleic acid (i.e., RNA, DNA, or cDNA) associated with a nucleic acid variation, over-expression, truncation, or fusions of RET from a tissue sample from the subject; wherein an increase in the amount of amplification product or labeled probe relative to a control indicates the presence of RET related cancer. It is further understood that to perform a PCR assay or hybridization assay on a nucleic acid to determine the presence of or an increase in RET as sample from a subject must be obtained and nucleic acid isolated. Therefore, also disclosed herein in are methods of diagnosing a RET related cancer comprising obtaining a tissue sample, isolating nucleic acid from the sample, performing PCR on the nucleic acid isolated from the tissue sample form a subject, and detecting the presence of or measuring the amount of nucleic acid associated with a nucleic acid variation, over-expression, truncation, or fusions of RET from a tissue sample from the subject; wherein an increase in the amount of amplification product or labeled probe relative to a control indicates the presence of RET related cancer.

### DEPDC1

Genome-wide cDNA microarray analysis originally identified over-expression of the DEP domain containing 1 gene (DEPDC1) gene in bladder cancer and later identified DEPDC1 as a molecular marker for breast cancer. Follow up investigations described a direct interaction between DEPDC1 and the zinc finger transcription factor ZNF224, a known transcriptional repressor of the NF-κB pathway responsible for inducing anti-apoptotic proteins in various human tumors. Co-immunoprecipitation studies using truncated forms of DEPDC1 further delineated the specific ZNF224 binding domain of DEPDC1 (FIG. 2B). Inhibiting the DEPDC1-ZNF224 interaction using a cell-permeable peptide induced apoptosis in bladder cancer cells in vitro and in vivo encouraging the use of these cell-permeable peptides as possible therapeutics.

The specific role DEPDC1 plays in NSCLC came to light when a second study used genome-wide expression profiling of 226 adenocarcinoma patients subdivided triple negative adenocarcinomas into a group A and group B cases. Group A had notably worse prognosis for relapse and death compared to group B directly correlating to the over-expression of the DEPDC1 in group A to a less favorable prognosis. On a separate but related note, a PrognoScan conducted in this study linked DEPDC1 expression to a poor prognosis in melanoma similar to what has been observed with RET expression potentially indicating like pathways of cellular hyper activation and proliferation by both oncogenes.

In one aspect, the methods disclosed herein can be accomplished through quantitative PCR assays, semi-quantitative PCR assays, or non-quantitative PCR assays. the methods can detect the presence or the overexpression of DEPDC1 for the diagnosing a DEPDC1 related cancer (such as, for example, a cancer occurring involving DEPDC1 overexpression). Thus, disclosed herein, in one aspect, are methods of detecting the presence of (i.e., diagnosing the presence) a DEPDC1 overexpression or a DEPDC1 fusion in a subject with a cancer comprising detecting the presence of or measuring the amount of nucleic acid (i.e., RNA, DNA, or cDNA) associated with a nucleic acid variation, over-expression, truncation, or fusions of DEPDC1 from a tissue sample from the subject; wherein an increase in the amount of amplification product or labeled probe relative to a control indicates the presence of DEPDC1 related cancer. It is further understood that to perform a PCR assay or hybridization assay on a nucleic acid to determine the presence of or an increase in DEPDC1 as sample from a subject must be obtained and nucleic acid isolated. Therefore, also disclosed herein in are methods of diagnosing a DEPDC1 related cancer comprising obtaining a tissue sample, isolating nucleic acid from the sample, performing PCR on the nucleic acid isolated from the tissue sample form a subject, and detecting the presence of or measuring the amount of nucleic acid associated with a nucleic acid variation, over-expression, truncation, or fusions of DEPDC1 from a tissue sample from the subject; wherein an increase in the amount of amplification product or labeled probe relative to a control indicates the presence of DEPDC1 related cancer.

KIF5B-RET and DEPDC1 are strong potential biomarkers for triple negative NSCLC for three main reasons: 1) Both KIF5B-RET and DEPDC1 are established drivers of triple negative NSCLC 2) known therapeutics exist for these two biomarkers, albeit not yet used for NSCLC, could provide efficacious treatments for triple negative NSCLC patients 3) the lack of constitutive expression of both KIF5B-RET and DEPDC1 in normal lung tissue allows for the quick development of highly sensitive and specific companion diagnostics.

Thus, in one aspect, disclosed herein are methods of diagnosing a RET or DEPDC1-related cancer comprising detecting the presence of or measuring the amount of nucleic acid associated with a nucleic acid variation, over-expression, truncation, or fusions of DEP domain containing 1 gene (DEPDC1) or RET from a tissue sample from the subject; wherein an increase in the amount of amplification product or labeled probe relative to a control indicates the presence of RET or DEPDC1 related cancer.

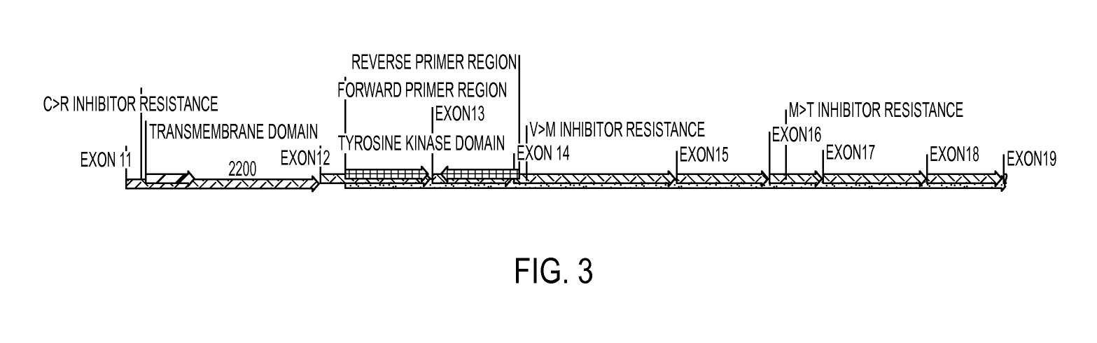

Chromosomal breaks in the RET gene have been observed in clinical specimens to occur 5′ of exon 11, leaving the transmembrane domain of the receptor intact. By targeting the detection downstream of the fusion break hot spot, the assay is not disrupted by variations in the fusion partners. In addition, the assay is designed to avoid inhibitor resistance mutations that have been shown to arise before or after treatment in other malignancies that can also arise in NSCLC patients treated with RET inhibitors. Both mutations observed clinically and in vitro from these RET inhibitor resistance studies were considered in the design to avoid loss of specificity and sensitivity when resistance mutations are present in the patient (FIG. 3).

In one aspect, the disclosed cancers can result from the fusion of an oncogene such as RET or DEPDC1 with a fusion partner or overexpression of RET or DEPDC1. Such chimeras include the known chimeras coiled coil domain containing 6-RET (CCDC6-RET), NCOA4-RET, and kinesin-1 heavy chain gene-RET (KIF5B-RET). Thus, in one aspect, the detection of RET or DEPDC1 fusion sequences or overexpression indicates the presence of a cancer. Therefore, disclosed herein are methods of diagnosing an RET or DEPDC1 related cancer in a subject comprising detecting the presence or measuring the expression level of nucleic acid (such as, for example mRNA, RNA, DNA, or cDNA) from a tissue sample from the subject; wherein the nucleic acid is specific to RET or DEPDC1 fusion; and wherein an increase in the amount of nucleic acid relative to a control indicates the presence of an RET or DEPDC1 related cancer.

The disclosed methods can also be used to diagnose cancers related to disregulation of RET or DEPDC1. The disregulated wild-type RET or DEPDC1 can result an increase/over-expression in wild-type RET or DEPDC1 being produced as well as the disregulation of the kinase catalytic activity of RET or DEPDC1. Therefore, disclosed herein are methods of diagnosing the presence of a cancer comprising detecting the presence or relative increase in the expression of mRNA relating to RET or DEPDC1 sequence. In one aspect, disclosed herein are methods of diagnosing a RET or DEPDC1 related cancer in a subject comprising measuring the expression level of DEPDC1 in a tissue sample form the subject, wherein increased expression of DEPDC1 relative to a cancer free control indicates the presence of a RET or DEPDC1-related cancer.

It is understood and herein contemplated that the cause of an RET or DEPDC1 related cancer can be due not only dysregulation of wild-type RET or DEPDC1 or known RET or DEPDC1 fusions, but one or more unidentified RET or DEPDC1 fusions. Methods that are only able to detect known fusions would be unable to detect previously unknown fusions or mutations of RET or DEPDC1. By detecting not only the presence of a RET Kinase or DEPDC1 3′ of any breakpoint the skilled artisan can determine if a cancer is due to unrelated to RET or DEPDC1 or due to a fusion of RET or DEPDC1 and a fusion partner. Because RET and DEPDC1 are typically not present in the lungs, detecting/measuring RET or DEPDC1 wild-type 5′ of any fusion breakpoint is unnecessary as any RET or DEPDC1 that increases relative to a control will be due to aberrant over-expression or a fusion.

However, in tissues where RET or DEPDC1 is present in some amount, additional specificity can be accomplished by further detecting/measuring the presence of wild-type RET or DEPDC1 5′ to any fusion breakpoint. In such methods, the presence of transcripts 5′ to a fusion breakpoint and 3′ to a fusion breakpoint (such as, for example the RET kinase domain) indicates wild-type expression and the level of expression relative to a control will determine if the cancer is due to over-expression of RET or DEPDC1. Where amplification product of sequences 3′ to a potential breakpoint is detected (such as for example RET kinase transcript or kinase activity) without corresponding amplification product to RET or DEPDC1 5′ to the fusion breakpoint, then a fusion of RET or DEPDC1 is present. Thus, in one aspect, disclosed herein are methods of diagnosing a RET or DEPDC1 related cancer in a subject comprising conducting a nucleic acid amplification process on a tissue sample from the subject and detecting the presence of or measuring the amount of nucleic acid associated with RET kinase domain, wild-type RET, or DEPDC1 in the tissue sample, wherein the presence or an increase in RET kinase wild-type RET, or DEPDC1 relative to a control indicates the presence of an RET or DEPDC1 related cancer.

It is understood and herein contemplated that after a tissue sample is removed from a subject, nucleic acid (e.g., DNA or RNA such as mRNA) can be isolated from the cells of the tissue. Thus, in a further aspect, the disclosed methods comprise obtaining a tissue sample and isolating nucleic acid from the tissue sample. For example, the methods can comprise taking a pulmonary tissue biopsy or sputum sample and isolating mRNA from the sample. It is further understood that where mRNA is isolated from the tissue sample, cDNA can be synthesized from the mRNA and PCR performed on the cDNA (for example, as part of an RT-PCR reaction). Thus, in one aspect, disclosed herein are methods of diagnosing a RET or DEPDC1 related cancer in a subject with a cancer, comprising obtaining a tissue sample from the subject, isolating nucleic acid (e.g., mRNA) from the tissue sample, conducting RT-PCR, real-time PCR, or real-time RT-PCR on the nucleic acid, and detecting the presence of or measuring the amount of nucleic acid associated with one of or a combination of wild-type RET (such as, for example the extracellular domain (ECD) of RET), RET kinase domain, or DEPDC1 in the tissue sample, wherein the PCR, RT-PCR, real-time PCR, or real-time RT-PCR reaction comprises the use of a forward and reverse primer pair that specifically hybridizes to a wild-type RET sequence (such as, for example, primers that hybridize to the extracellular domain (ECD) of RET, such as SEQ ID NOs: 6 and SEQ ID NO: 7) and/or a forward and reverse primer pair that specifically hybridizes to a wild-type RET kinase domain sequence (such as, for example, SEQ ID NOs: 3, 4, 12, 13, 15, and 16) or the PCR, RT-PCR, real-time PCR, or real-time RT-PCR reaction comprises the use of a forward and reverse primer pair that specifically hybridizes to a DEPDC1 sequence (such as, for example primers that hybridize to DEPDC1, such as SEQ ID NO: 9, 10, 18, 19, 21, 22, 24, and 25) and detecting the presence of or measuring the amount of nucleic acid associated with one or a combination of both wild-type RET and RET kinase domain in the tissue sample or DEPDC1 in a tissue sample, wherein an increase in amplicon relative to a normal control or the presence of an amplicon indicates the that the subject has a RET or DEPDC1 related cancer. Also disclosed are methods for diagnosing a RET or DEPDC1-related cancer in a subject with a cancer comprising obtaining a tissue sample from the subject, isolating nucleic acid from the tissue sample, wherein the nucleic acid from the tissue sample is RNA, wherein the method further comprises synthesizing cDNA from the RNA sample, conducting PCR on the cDNA; and detecting the presence of or measuring the amount of nucleic acid associated with one or a combination of both wild-type RET and RET kinase domain or DEPDC1 in the tissue sample, wherein the presence or an increase in amplicon relative to a normal control or the presence of an amplicon indicates the that the subject has a RET or DEPDC1 related cancer.

It is understood and herein contemplated that the disclosed methods of diagnosis and determination of susceptibility or resistance to RET or DEPDC1 inhibitor treatment can be used not only on subjects that have not previously been diagnosed with a cancer to identify that the subject has cancer, but specifically on subjects having been previously diagnosed with a cancer and the method used to diagnose that the cancer is specifically RET or DEPDC1 related or susceptible to treatment and thus not to diagnose a cancer but to determine if a known cancer in a subject is RET or DEPDC1-related or susceptible to treatment with a RET or DEPDC1 inhibitor.

mRNA Detection and Quantification

The methods disclosed herein relate to the detection of nucleic acid variation in the form of, for example, point mutations and truncations, or the detection of expression of RET or DEPDC1 fusions, aberrant wild-type RET or DEPDC1 expression (such as over-expression), or expression of RET or DEPDC1 truncation mutants. For these latter expression level detections, the methods comprise detecting either the abundance or presence of mRNA, or both. Thus, disclosed herein are methods and compositions for diagnosing an RET or DEPDC1-related cancer in a subject comprising measuring the presence or level of mRNA from a tissue sample from the subject; wherein an increase in the amount of mRNA relative to a control indicates the presence of an RET or DEPDC1 related cancer.

A number of widely used procedures exist for detecting and determining the abundance of a particular mRNA in a total or poly(A) RNA sample. For example, specific mRNAs can be detected using Northern blot analysis, nuclease protection assays (NPA), in situ hybridization, or reverse transcription-polymerase chain reaction (RT-PCR), real-time PCR, real-time RT-PCR, and microarray.

Each of these techniques can be used to detect specific RNAs and to precisely determine their expression level. In general, Northern analysis is the only method that provides information about transcript size, whereas NPAs are the easiest way to simultaneously examine multiple messages. In situ hybridization is used to localize expression of a particular gene within a tissue or cell type, and real-time PCR, RT-PCR, and real-time RT-PCR are the most sensitive method for detecting and quantitating gene expression.

Real-time PCR, RT-PCR, and real-time RT-PCR allow for the detection of the RNA transcript of any gene, regardless of the scarcity of the starting material or relative abundance of the specific mRNA. In RT-PCR, an RNA template is copied into a complementary DNA (cDNA) using a retroviral reverse transcriptase. The cDNA is then amplified exponentially by PCR using a DNA polymerase. The reverse transcription and PCR reactions can occur in the same or difference tubes. RT-PCR is somewhat tolerant of degraded RNA. As long as the RNA is intact within the region spanned by the primers, the target will be amplified.

Relative quantitative RT-PCR involves amplifying an internal control simultaneously with the gene of interest. The internal control is used to normalize the samples. Once normalized, direct comparisons of relative abundance of a specific mRNA can be made across the samples. It is crucial to choose an internal control with a constant level of expression across all experimental samples (i.e., not affected by experimental treatment). Commonly used internal controls (e.g., GAPDH, β-actin, cyclophilin) often vary in expression and, therefore, may not be appropriate internal controls. Additionally, most common internal controls are expressed at much higher levels than the mRNA being studied. For relative RT-PCR results to be meaningful, all products of the PCR reaction must be analyzed in the linear range of amplification. This becomes difficult for transcripts of widely different levels of abundance.

Competitive RT-PCR is used for absolute quantitation. This technique involves designing, synthesizing, and accurately quantitating a competitor RNA that can be distinguished from the endogenous target by a small difference in size or sequence. Known amounts of the competitor RNA are added to experimental samples and RT-PCR is performed. Signals from the endogenous target are compared with signals from the competitor to determine the amount of target present in the sample.

Disclosed herein in one aspect are methods of diagnosing an RET or DEPDC1 related cancer in a subject comprising conducting real-time PCR, RT-PCR, real-time RT-PCR, or other PCR reaction on nucleic acid such as, for example, mRNA, DNA, or cDNA from a tissue sample from the subject; wherein the polymerase chain reaction comprises a reverse primer capable of specifically hybridizing to one or more RET or DEPDC1 sequences and at least one forward primer; and wherein an increase in the amount of amplification product relative to a control indicates the presence of an RET or DEPDC1 related cancer. Also disclosed herein are methods of diagnosing an RET or DEPDC1 related cancer in a subject comprising conducting FISH on a tissue sample from the subject; wherein the polymerase chain reaction comprises probes capable of specifically hybridizing to one or more RET or DEPDC1 sequences on separate sides of a RET or DEPDC1 fusion breakpoint; and wherein a disrupted gene locus indicated by separated probes indicates the presence of an RET or DEPDC1 related cancer. Examples of probes for use in this assay include those found on Table 2.

As the disclosed methods can be used to detect wild-type RET or DEPDC1, RET or DEPDC1 fusions, RET or DEPDC1 over-expression, and RET or DEPDC1 kinase domain activity, also disclosed herein are methods of diagnosing an RET or DEPDC1 related cancer or detecting the dysregulation of an RET or DEPDC1 kinase in a subject comprising conducting a RT-PCR reaction on mRNA from a tissue sample from the subject; wherein the reverse transcription polymerase chain reaction (RT-PCR) comprises one primer pair capable of specifically hybridizing to a RET kinase sequences (such as, for example SEQ ID NOs: 3, 4, 12, 13, 15, and 16) and/or at least one primer pair capable of specifically hybridizing to RET 5′ of any fusion breakpoint (i.e., an external wild-type RET site such as, for example, SEQ ID NOs: 6 and 7) or at least on primer pair capable of specifically hybridizing to DEPDC1 (such as, for example SEQ ID NOs: 9, 10, 18, 19, 21, 22, 24, and 25) and detecting the presence of or increase in the amplicon, wherein the presence of RET OR DEPDC11 sequences in the amplicon from the first reaction indicates that presence of a RET OR DEPDC11 related cancer (i.e, overexpression or fusion is present). It is further contemplated when both a wild-type RET primer pair and a RET kinase primer pair are used, the primer pairs can be used in sequential reactions where one primer pair can be used to amplify the amplicon o the first primer pair used. For example, disclosed herein are methods of diagnosing an RET related cancer or detecting the dysregulation of an RET kinase in a subject comprising conducting a first RT-PCR reaction on mRNA from a tissue sample from the subject; wherein the reverse transcription polymerase chain reaction (RT-PCR) comprises one primer pair capable of specifically hybridizing to a RET kinase sequences (such as, for example SEQ ID NOs: 3, 4, 12, 13, 15, and 16) and/or at least one primer pair capable of specifically hybridizing to RET 5′ of any fusion breakpoint (i.e., an external wild-type RET site such as, for example, SEQ ID NOs: 6 and 7) and detecting the presence of or amplifying the amplicon from the first reaction using one or more primers that specifically hybridize to RET OR DEPDC11 sequences 3′ of the fusion breakpoint, wherein the presence of RET OR DEPDC11 sequences in the amplicon from the first reaction indicates that presence of a RET OR DEPDC11 fusion.

Real-Time PCR

Real-time PCR monitors the fluorescence emitted during the reaction as an indicator of amplicon production during each PCR cycle (i.e., in real time) as opposed to the endpoint detection. The real-time progress of the reaction can be viewed in some systems. Real-time PCR does not detect the size of the amplicon and thus does not allow the differentiation between DNA and cDNA amplification, however, it is not influenced by non-specific amplification unless SYBR Green is used. Real-time PCR quantitation eliminates post-PCR processing of PCR products. This helps to increase throughput and reduce the chances of carryover contamination. Real-time PCR also offers a wide dynamic range of up to 10⁷-fold. Dynamic range of any assay determines how much target concentration can vary and still be quantified. A wide dynamic range means that a wide range of ratios of target and normaliser can be assayed with equal sensitivity and specificity. It follows that the broader the dynamic range, the more accurate the quantitation. When combined with RT-PCR, a real-time RT-PCR reaction reduces the time needed for measuring the amount of amplicon by providing for the visualization of the amplicon as the amplification process is progressing.

The real-time PCR system is based on the detection and quantitation of a fluorescent reporter. This signal increases in direct proportion to the amount of PCR product in a reaction. By recording the amount of fluorescence emission at each cycle, it is possible to monitor the PCR reaction during exponential phase where the first significant increase in the amount of PCR product correlates to the initial amount of target template. The higher the starting copy number of the nucleic acid target, the sooner a significant increase in fluorescence is observed. A significant increase in fluorescence above the baseline value measured during the 3-15 cycles can indicate the detection of accumulated PCR product.

A fixed fluorescence threshold is set significantly above the baseline that can be altered by the operator. The parameter C_{T} (threshold cycle) is defined as the cycle number at which the fluorescence emission exceeds the fixed threshold.

There are three main fluorescence-monitoring systems for DNA amplification: (1) hydrolysis probes; (2) hybridising probes; and (3) DNA-binding agents. Hydrolysis probes include TaqMan probes, molecular beacons and scorpions. They use the fluorogenic 5′ exonuclease activity of Taq polymerase to measure the amount of target sequences in cDNA samples.

TaqMan probes are oligonucleotides longer than the primers (20-30 bases long with a Tm value of 10° C. higher) that contain a fluorescent dye usually on the 5′ base, and a quenching dye (usually TAMRA) typically on the 3′ base. When irradiated, the excited fluorescent dye transfers energy to the nearby quenching dye molecule rather than fluorescing (this is called FRET=Förster or fluorescence resonance energy transfer). Thus, the close proximity of the reporter and quencher prevents emission of any fluorescence while the probe is intact. TaqMan probes are designed to anneal to an internal region of a PCR product. When the polymerase replicates a template on which a TaqMan probe is bound, its 5′ exonuclease activity cleaves the probe. This ends the activity of quencher (no FRET) and the reporter dye starts to emit fluorescence which increases in each cycle proportional to the rate of probe cleavage. Accumulation of PCR products is detected by monitoring the increase in fluorescence of the reporter dye (note that primers are not labelled). TaqMan assay uses universal thermal cycling parameters and PCR reaction conditions. Because the cleavage occurs only if the probe hybridises to the target, the origin of the detected fluorescence is specific amplification. The process of hybridisation and cleavage does not interfere with the exponential accumulation of the product. One specific requirement for fluorogenic probes is that there be no G at the 5′ end. A ‘G’ adjacent to the reporter dye can quench reporter fluorescence even after cleavage.

Molecular beacons also contain fluorescent (FAM, TAMRA, TET, ROX) and quenching dyes (typically DABCYL) at either end but they are designed to adopt a hairpin structure while free in solution to bring the fluorescent dye and the quencher in close proximity for FRET to occur. They have two arms with complementary sequences that form a very stable hybrid or stem. The close proximity of the reporter and the quencher in this hairpin configuration suppresses reporter fluorescence. When the beacon hybridises to the target during the annealing step, the reporter dye is separated from the quencher and the reporter fluoresces (FRET does not occur). Molecular beacons remain intact during PCR and must rebind to target every cycle for fluorescence emission. This will correlate to the amount of PCR product available. All real-time PCR chemistries allow detection of multiple DNA species (multiplexing) by designing each probe/beacon with a spectrally unique fluor/quench pair as long as the platform is suitable for melting curve analysis if SYBR green is used. By multiplexing, the target(s) and endogenous control can be amplified in single tube.

With Scorpion probes, sequence-specific priming and PCR product detection is achieved using a single oligonucleotide. The Scorpion probe maintains a stem-loop configuration in the unhybridised state. The fluorophore is attached to the 5′ end and is quenched by a moiety coupled to the 3′ end. The 3′ portion of the stem also contains sequence that is complementary to the extension product of the primer. This sequence is linked to the 5′ end of a specific primer via a non-amplifiable monomer. After extension of the Scorpion primer, the specific probe sequence is able to bind to its complement within the extended amplicon thus opening up the hairpin loop. This prevents the fluorescence from being quenched and a signal is observed.

Another alternative is the double-stranded DNA binding dye chemistry, which quantitates the amplicon production (including non-specific amplification and primer-dimer complex) by the use of a non-sequence specific fluorescent intercalating agent (SYBR-green I or ethidium bromide). It does not bind to ssDNA. SYBR green is a fluorogenic minor groove binding dye that exhibits little fluorescence when in solution but emits a strong fluorescent signal upon binding to double-stranded DNA. Disadvantages of SYBR green-based real-time PCR include the requirement for extensive optimisation. Furthermore, non-specific amplifications require follow-up assays (melting point curve or dissociation analysis) for amplicon identification. The method has been used in HFE-C282Y genotyping. Another controllable problem is that longer amplicons create a stronger signal (if combined with other factors, this may cause CDC camera saturation, see below). Normally SYBR green is used in singleplex reactions, however when coupled with melting point analysis, it can be used for multiplex reactions.

The threshold cycle or the C_{T} value is the cycle at which a significant increase in ΔRn is first detected (for definition of ΔRn, see below). The threshold cycle is when the system begins to detect the increase in the signal associated with an exponential growth of PCR product during the log-linear phase. This phase provides the most useful information about the reaction (certainly more important than the end-point). The slope of the log-linear phase is a reflection of the amplification efficiency. The efficiency (Eff) of the reaction can be calculated by the formula: Eff=10^{(-1/slope)}−1. The efficiency of the PCR should be 90-100% (3.6>slope>3.1). A number of variables can affect the efficiency of the PCR. These factors include length of the amplicon, secondary structure and primer quality. Although valid data can be obtained that fall outside of the efficiency range, the qRT-PCR should be further optimised or alternative amplicons designed. For the slope to be an indicator of real amplification (rather than signal drift), there has to be an inflection point. This is the point on the growth curve when the log-linear phase begins. It also represents the greatest rate of change along the growth curve. (Signal drift is characterised by gradual increase or decrease in fluorescence without amplification of the product.) The important parameter for quantitation is the C_{T}. The higher the initial amount of genomic DNA, the sooner accumulated product is detected in the PCR process, and the lower the C_{T} value. The threshold should be placed above any baseline activity and within the exponential increase phase (which looks linear in the log transformation). Some software allows determination of the cycle threshold (C_{T}) by a mathematical analysis of the growth curve. This provides better run-to-run reproducibility. A C_{T} value of 40 means no amplification and this value cannot be included in the calculations. Besides being used for quantitation, the C_{T} value can be used for qualitative analysis as a pass/fail measure.

Multiplex TaqMan assays can be performed using multiple dyes with distinct emission wavelengths. Available dyes for this purpose are FAM, TET, VIC and JOE (the most expensive). TAMRA is reserved as the quencher on the probe and ROX as the passive reference. For best results, the combination of FAM (target) and VIC (endogenous control) is recommended (they have the largest difference in emission maximum) whereas JOE and VIC should not be combined. It is important that if the dye layer has not been chosen correctly, the machine will still read the other dye's spectrum. For example, both VIC and FAM emit fluorescence in a similar range to each other and when doing a single dye, the wells should be labelled correctly. In the case of multiplexing, the spectral compensation for the post run analysis should be turned on (on ABI 7700: Instrument/Diagnostics/Advanced Options/Miscellaneous). Activating spectral compensation improves dye spectral resolution.

Thus, disclosed herein in one aspect are methods of diagnosing RET or DEPDC1-related cancer in a subject comprising conducting an RT-PCR, real-time PCR, or real-time RT-PCR reaction on nucleic acid (such as, for example RNA, mRNA, DNA, cDNA) from a tissue sample from the subject; wherein the real-time PCR or reverse transcription polymerase chain reaction (RT-PCR) or real-time RT-PCR reaction comprises a reverse primer capable of specifically hybridizing to one or more RET or DEPDC1 sequences and at least one forward primer; and wherein an increase in the amount of amplification product relative to a control indicates the presence of an RET or DEPDC1-related cancer. In another aspect, disclosed herein are methods of diagnosing a RET or DEPDC1-related cancer wherein the nucleic acid is measured by measuring mRNA levels by conducting a first reverse transcription polymerase chain reaction (RT-PCR) or real time polymerase chain reaction on the sample.

Northern analysis is the easiest method for determining transcript size, and for identifying alternatively spliced transcripts and multigene family members. It can also be used to directly compare the relative abundance of a given message between all the samples on a blot. The Northern blotting procedure is straightforward and provides opportunities to evaluate progress at various points (e.g., intactness of the RNA sample and how efficiently it has transferred to the membrane). RNA samples are first separated by size via electrophoresis in an agarose gel under denaturing conditions. The RNA is then transferred to a membrane, crosslinked and hybridized with a labeled probe. Nonisotopic or high specific activity radiolabeled probes can be used including random-primed, nick-translated, or PCR-generated DNA probes, in vitro transcribed RNA probes, and oligonucleotides. Additionally, sequences with only partial homology (e.g., cDNA from a different species or genomic DNA fragments that might contain an exon) may be used as probes.

The Nuclease Protection Assay (NPA) (including both ribonuclease protection assays and S1 nuclease assays) is a sensitive method for the detection and quantitation of specific mRNAs. The basis of the NPA is solution hybridization of an antisense probe (radiolabeled or nonisotopic) to an RNA sample. After hybridization, single-stranded, unhybridized probe and RNA are degraded by nucleases. The remaining protected fragments are separated on an acrylamide gel. Solution hybridization is typically more efficient than membrane-based hybridization, and it can accommodate up to 100 μg of sample RNA, compared with the 20-30 μg maximum of blot hybridizations. NPAs are also less sensitive to RNA sample degradation than Northern analysis since cleavage is only detected in the region of overlap with the probe (probes are usually about 100-400 bases in length).

NPAs are the method of choice for the simultaneous detection of several RNA species. During solution hybridization and subsequent analysis, individual probe/target interactions are completely independent of one another. Thus, several RNA targets and appropriate controls can be assayed simultaneously (up to twelve have been used in the same reaction), provided that the individual probes are of different lengths. NPAs are also commonly used to precisely map mRNA termini and intron/exon junctions.

In situ hybridization (ISH) is a powerful and versatile tool for the localization of specific mRNAs in cells or tissues. Unlike Northern analysis and nuclease protection assays, ISH does not require the isolation or electrophoretic separation of RNA. Hybridization of the probe takes place within the cell or tissue. Since cellular structure is maintained throughout the procedure, ISH provides information about the location of mRNA within the tissue sample.

The procedure begins by fixing samples in neutral-buffered formalin, and embedding the tissue in paraffin. The samples are then sliced into thin sections and mounted onto microscope slides. (Alternatively, tissue can be sectioned frozen and post-fixed in paraformaldehyde.) After a series of washes to dewax and rehydrate the sections, a Proteinase K digestion is performed to increase probe accessibility, and a labeled probe is then hybridized to the sample sections. Radiolabeled probes are visualized with liquid film dried onto the slides, while nonisotopically labeled probes are conveniently detected with colorimetric or fluorescent reagents.

DNA Detection and Quantification

The methods disclosed herein relate to the detection of nucleic acid variation in the form of, for example, point mutations and truncations, or the detection of expression of RET or DEPDC1 fusions, aberrant wild-type RET or DEPDC1 expression, or expression of RET or DEPDC1 truncation mutants. For these latter expression level detections, the methods comprise detecting either the abundance or presence of mRNA, or both. Alternatively, detection can directed to the abundance or presence of DNA, for example, cDNA. Thus, disclosed herein are methods and compositions for diagnosing a RET or DEPDC1-related cancer in a subject comprising measuring the presence or level of DNA from a tissue sample from the subject; wherein an increase in the amount of DNA relative to a control indicates the presence of an RET or DEPDC1-related cancer.

A number of widely used procedures exist for detecting and determining the abundance of a particular DNA in a sample. For example, the technology of PCR permits amplification and subsequent detection of minute quantities of a target nucleic acid. Details of PCR are well described in the art, including, for example, U.S. Pat. No. 4,683,195 to Mullis et al., U.S. Pat. No. 4,683,202 to Mullis and U.S. Pat. No. 4,965,188 to Mullis et al. Generally, oligonucleotide primers are annealed to the denatured strands of a target nucleic acid, and primer extension products are formed by the polymerization of deoxynucleoside triphosphates by a polymerase. A typical PCR method involves repetitive cycles of template nucleic acid denaturation, primer annealing and extension of the annealed primers by the action of a thermostable polymerase. The process results in exponential amplification of the target nucleic acid, and thus allows the detection of targets existing in very low concentrations in a sample. It is understood and herein contemplated that there are variant PCR methods known in the art that may also be utilized in the disclosed methods, for example, Quantitative PCR (QPCR); microarrays, real-time PCR; hot start PCR; nested PCR; allele-specific PCR; and Touchdown PCR.

Microarrays

An array is an orderly arrangement of samples, providing a medium for matching known and unknown DNA samples based on base-pairing rules and automating the process of identifying the unknowns. An array experiment can make use of common assay systems such as microplates or standard blotting membranes, and can be created by hand or make use of robotics to deposit the sample. In general, arrays are described as macroarrays or microarrays, the difference being the size of the sample spots. Macroarrays contain sample spot sizes of about 300 microns or larger and can be easily imaged by existing gel and blot scanners. The sample spot sizes in microarray can be 300 microns or less, but typically less than 200 microns in diameter and these arrays usually contains thousands of spots. Microarrays require specialized robotics and/or imaging equipment that generally are not commercially available as a complete system. Terminologies that have been used in the literature to describe this technology include, but not limited to: biochip, DNA chip, DNA microarray, GENE CHIP® (Affymetrix, Inc which refers to its high density, oligonucleotide-based DNA arrays), and gene array.

DNA microarrays, or DNA chips are fabricated by high-speed robotics, generally on glass or nylon substrates, for which probes with known identity are used to determine complementary binding, thus allowing massively parallel gene expression and gene discovery studies. An experiment with a single DNA chip can provide information on thousands of genes simultaneously. It is herein contemplated that the disclosed microarrays can be used to monitor gene expression, disease diagnosis, gene discovery, drug discovery (pharmacogenomics), and toxicological research or toxicogenomics.

There are two variants of the DNA microarray technology, in terms of the property of arrayed DNA sequence with known identity. Type I microarrays comprise a probe cDNA (500˜5,000 bases long) that is immobilized to a solid surface such as glass using robot spotting and exposed to a set of targets either separately or in a mixture. This method is traditionally referred to as DNA microarray. With Type I microarrays, localized multiple copies of one or more polynucleotide sequences, preferably copies of a single polynucleotide sequence are immobilized on a plurality of defined regions of the substrate's surface. A polynucleotide refers to a chain of nucleotides ranging from 5 to 10,000 nucleotides. These immobilized copies of a polynucleotide sequence are suitable for use as probes in hybridization experiments.

To prepare beads coated with immobilized probes, beads are immersed in a solution containing the desired probe sequence and then immobilized on the beads by covalent or noncovalent means. Alternatively, when the probes are immobilized on rods, a given probe can be spotted at defined regions of the rod. Typical dispensers include a micropipette delivering solution to the substrate with a robotic system to control the position of the micropipette with respect to the substrate. There can be a multiplicity of dispensers so that reagents can be delivered to the reaction regions simultaneously. In one embodiment, a microarray is formed by using ink jet technology based on the piezoelectric effect, whereby a narrow tube containing a liquid of interest, such as oligonucleotide synthesis reagents, is encircled by an adapter. An electric charge sent across the adapter causes the adapter to expand at a different rate than the tube and forces a small drop of liquid onto a substrate.

Samples may be any sample containing polynucleotides (polynucleotide targets) of interest and obtained from any bodily fluid (blood, urine, saliva, phlegm, gastric juices, etc.), cultured cells, biopsies, or other tissue preparations. DNA or RNA can be isolated from the sample according to any of a number of methods well known to those of skill in the art. In one embodiment, total RNA is isolated using the TRIzol total RNA isolation reagent (Life Technologies, Inc., Rockville, Md.) and RNA is isolated using oligo d(T) column chromatography or glass beads. After hybridization and processing, the hybridization signals obtained should reflect accurately the amounts of control target polynucleotide added to the sample.

The plurality of defined regions on the substrate can be arranged in a variety of formats. For example, the regions may be arranged perpendicular or in parallel to the length of the casing. Furthermore, the targets do not have to be directly bound to the substrate, but rather can be bound to the substrate through a linker group. The linker groups may typically vary from about 6 to 50 atoms long. Linker groups include ethylene glycol oligomers, diamines, diacids and the like. Reactive groups on the substrate surface react with one of the terminal portions of the linker to bind the linker to the substrate. The other terminal portion of the linker is then functionalized for binding the probes.

Sample polynucleotides may be labeled with one or more labeling moieties to allow for detection of hybridized probe/target polynucleotide complexes. The labeling moieties can include compositions that can be detected by spectroscopic, photochemical, biochemical, bioelectronic, immunochemical, electrical, optical or chemical means. The labeling moieties include radioisotopes, such as ³²P, ³³P or ³⁵S, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, biotin, and the like.

Labeling can be carried out during an amplification reaction, such as polymerase chain reaction and in vitro or in vivo transcription reactions. Alternatively, the labeling moiety can be incorporated after hybridization once a probe-target complex his formed. In one embodiment, biotin is first incorporated during an amplification step as described above. After the hybridization reaction, unbound nucleic acids are rinsed away so that the only biotin remaining bound to the substrate is that attached to target polynucleotides that are hybridized to the polynucleotide probes. Then, an avidin-conjugated fluorophore, such as avidin-phycoerythrin, that binds with high affinity to biotin is added.

Hybridization causes a polynucleotide probe and a complementary target to form a stable duplex through base pairing. Hybridization methods are well known to those skilled in the art Stringent conditions for hybridization can be defined by salt concentration, temperature, and other chemicals and conditions. Varying additional parameters, such as hybridization time, the concentration of detergent (sodium dodecyl sulfate, SDS) or solvent (formamide), and the inclusion or exclusion of carrier DNA, are well known to those skilled in the art. Additional variations on these conditions will be readily apparent to those skilled in the art.

Methods for detecting complex formation are well known to those skilled in the art. In one embodiment, the polynucleotide probes are labeled with a fluorescent label and measurement of levels and patterns of complex formation is accomplished by fluorescence microscopy, preferably confocal fluorescence microscopy. An argon ion laser excites the fluorescent label, emissions are directed to a photomultiplier and the amount of emitted light detected and quantitated. The detected signal should be proportional to the amount of probe/target polynucleotide complex at each position of the microarray. The fluorescence microscope can be associated with a computer-driven scanner device to generate a quantitative two-dimensional image of hybridization intensities. The scanned image is examined to determine the abundance/expression level of each hybridized target polynucleotide.

In a differential hybridization experiment, polynucleotide targets from two or more different biological samples are labeled with two or more different fluorescent labels with different emission wavelengths. Fluorescent signals are detected separately with different photomultipliers set to detect specific wavelengths. The relative abundances/expression levels of the target polynucleotides in two or more samples is obtained. Typically, microarray fluorescence intensities can be normalized to take into account variations in hybridization intensities when more than one microarray is used under similar test conditions. In one embodiment, individual polynucleotide probe/target complex hybridization intensities are normalized using the intensities derived from internal normalization controls contained on each microarray.

Type II microarrays comprise an array of oligonucleotides (20˜80-mer oligos) or peptide nucleic acid (PNA) probes that is synthesized either in situ (on-chip) or by conventional synthesis followed by on-chip immobilization. The array is exposed to labeled sample DNA, hybridized, and the identity/abundance of complementary sequences are determined. This method, “historically” called DNA chips, was developed at Affymetrix, Inc., which sells its photolithographically fabricated products under the GeneChip® trademark.

The basic concept behind the use of Type II arrays for gene expression is simple: labeled cDNA or cRNA targets derived from the mRNA of an experimental sample are hybridized to nucleic acid probes attached to the solid support. By monitoring the amount of label associated with each DNA location, it is possible to infer the abundance of each mRNA species represented. Although hybridization has been used for decades to detect and quantify nucleic acids, the combination of the miniaturization of the technology and the large and growing amounts of sequence information, have enormously expanded the scale at which gene expression can be studied.

Microarray manufacturing can begin with a 5-inch square quartz wafer. Initially the quartz is washed to ensure uniform hydroxylation across its surface. Because quartz is naturally hydroxylated, it provides an excellent substrate for the attachment of chemicals, such as linker molecules, that are later used to position the probes on the arrays.

The wafer is placed in a bath of silane, which reacts with the hydroxyl groups of the quartz, and forms a matrix of covalently linked molecules. The distance between these silane molecules determines the probes' packing density, allowing arrays to hold over 500,000 probe locations, or features, within a mere 1.28 square centimeters. Each of these features harbors millions of identical DNA molecules. The silane film provides a uniform hydroxyl density to initiate probe assembly. Linker molecules, attached to the silane matrix, provide a surface that may be spatially activated by light.

Probe synthesis occurs in parallel, resulting in the addition of an A, C, T, or G nucleotide to multiple growing chains simultaneously. To define which oligonucleotide chains will receive a nucleotide in each step, photolithographic masks, carrying 18 to 20 square micron windows that correspond to the dimensions of individual features, are placed over the coated wafer. The windows are distributed over the mask based on the desired sequence of each probe. When ultraviolet light is shone over the mask in the first step of synthesis, the exposed linkers become deprotected and are available for nucleotide coupling.

Once the desired features have been activated, a solution containing a single type of deoxynucleotide with a removable protection group is flushed over the wafer's surface. The nucleotide attaches to the activated linkers, initiating the synthesis process.

Although each position in the sequence of an oligonucleotide can be occupied by 1 of 4 nucleotides, resulting in an apparent need for 25×4, or 100, different masks per wafer, the synthesis process can be designed to significantly reduce this requirement. Algorithms that help minimize mask usage calculate how to best coordinate probe growth by adjusting synthesis rates of individual probes and identifying situations when the same mask can be used multiple times.

Some of the key elements of selection and design are common to the production of all microarrays, regardless of their intended application. Strategies to optimize probe hybridization, for example, are invariably included in the process of probe selection. Hybridization under particular pH, salt, and temperature conditions can be optimized by taking into account melting temperatures and using empirical rules that correlate with desired hybridization behaviors.

To obtain a complete picture of a gene's activity, some probes are selected from regions shared by multiple splice or polyadenylation variants. In other cases, unique probes that distinguish between variants are favored. Inter-probe distance is also factored into the selection process.

A different set of strategies is used to select probes for genotyping arrays that rely on multiple probes to interrogate individual nucleotides in a sequence. The identity of a target base can be deduced using four identical probes that vary only in the target position, each containing one of the four possible bases.

Alternatively, the presence of a consensus sequence can be tested using one or two probes representing specific alleles. To genotype heterozygous or genetically mixed samples, arrays with many probes can be created to provide redundant information, resulting in unequivocal genotyping. In addition, generic probes can be used in some applications to maximize flexibility. Some probe arrays, for example, allow the separation and analysis of individual reaction products from complex mixtures, such as those used in some protocols to identify single nucleotide polymorphisms (SNPs).

Nested PCR

The disclosed methods can further utilize nested PCR. Nested PCR increases the specificity of DNA amplification, by reducing background due to non-specific amplification of DNA. Two sets of primers are being used in two successive PCRs. In the first reaction, one pair of primers is used to generate DNA products, which besides the intended target, may still consist of non-specifically amplified DNA fragments. The product(s) are then used in a second PCR with a set of primers whose binding sites are completely or partially different from and located 3′ of each of the primers used in the first reaction. Nested PCR is often more successful in specifically amplifying long DNA fragments than conventional PCR, but it requires more detailed knowledge of the target sequences.

Thus, disclosed herein in one aspect are methods of diagnosing RET or DEPDC1 related cancer in a subject comprising conducting a PCR reaction on DNA from a tissue sample from the subject; wherein the PCR reaction comprises a reverse primer capable of specifically hybridizing to one or more RET or DEPDC1 sequences and at least one forward primer; and wherein an increase in the amount of amplification product relative to a control indicates the presence of an RET or DEPDC1 related cancer.

### Primers and Probes

As used herein, “primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

As used herein, “probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Disclosed are compositions including primers and probes, which are capable of interacting with the disclosed nucleic acids such as SEQ ID NO: 1 or SEQ ID NO: 2 or their complement such as those listed in Table 2 (e.g., SEQ ID NOs: 3, 4, 5, 6, 7, 8, 9, 10, 11, 12, 13, 14, 15, 116, 17, 18, 19, 20, 21, 22, 23, 24, 25, or 26)(SEQ ID NOs: 3, 4, 6, 7, 12, 13, 15, and 16 are primers that interact with SEQ ID NO: 1; SEQ ID NOs: 9, 10, 18, 19, 21, 22, 24, and 25 are primers that interact with SEQ ID NO: 2; SEQ ID NOs: 5, 8, 14, and 17 are probes that interact with SEQ ID NO: 1; and SEQ ID NOs: 11, 20, 23, and 26 are probes that interact with SEQ ID NO: 2). In certain embodiments the primers are used to support nucleic acid extension reactions, nucleic acid replication reactions, and/or nucleic acid amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are disclosed. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids. As an example of the use of primers, one or more primers can be used to create extension products from and templated by a first nucleic acid.

The size of the primers or probes for interaction with the nucleic acids can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primers for the nucleic acid of interest typically will be used to produce extension products and/or other replicated or amplified products that contain a region of the nucleic acid of interest. The size of the product can be such that the size can be accurately determined to within 3, or 2 or 1 nucleotides.

In certain embodiments the product can be, for example, at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product can be, for example, less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

It is understood and herein contemplated that the disclosed RT-PCR and PCR reactions require forward and reverse primers to form a primer pair. Thus, in one aspect, disclosed herein are methods of diagnosing an RET or DEPDC1-related cancer, wherein the nucleic acid is measured by measuring mRNA levels by conducting a first reverse transcription polymerase chain reaction (RT-PCR) or real time polymerase chain reaction on the sample, and wherein the RT-PCR, real-time PCR, or real-time RT-PCR reaction comprises the use of a reverse primer capable of specifically hybridizing to one or more RET or DEPDC1 target sequences and at least one or more forward primers.

Herein disclosed, the forward primer can be selected from the group consisting of For example, where RET-related cancers are being diagnosed the forward primer can be an intracellular RET primer such as, for example, 5′ TGGCCGTGAAGATGCTGAAAGAGA 3′(SEQ ID NO: 3) or SEQ ID NO: 12 or 15 or an extracellular wild-type RET primer from a region of non-homology such as, for example, 5′ CCAGTACCTACTCCCTCTCCGTGA 3′ (SEQ ID NO: 6). The reverse primer can be, for example, 5′TGATGACATGTGGGTGGTTGACCT 3′ (SEQ ID NO: 4) or SEQ ID NO: 13 or 16. Alternatively, the reverse primer can be TGTACTGGACGTTGATGCCACTGA (SEQ ID NO: 7). In situations where the diagnosis of DEPDC1-related cancers is the goal, the forward primer and reverse primers can be any primer pair that can be used to detect DEPDC1. For example, in one aspect, the forward primer can be 5′ TGCAATGGGTACGAGGTCACTGAT 3′ (SEQ ID NO: 9) or SEQ ID NO: 18, 21, or 24 and the reverse primer can be 5′ CCAGCAAGAAGCTCATCAAGATCC 3′ (SEQ ID NO: 10) or SEQ ID NO: 19, 22, or 25. It is understood that the methods comprise at least one forward primer and a reverse primer.

In one aspect, disclosed herein are methods of diagnosing a RET or DEPDC1 related cancer in a subject comprising conducting an PCR, real-time PCR, RT-PCR, real-time RT-PCR reaction on mRNA, DNA, or cDNA from a tissue sample from the subject; wherein the reverse primer capable of specifically hybridizing to one or more RET or DEPDC1 sequences and at least one forward primer; wherein the forward primer is selected from the group consisting of a KIF5B primer that hybridizes 5′ to a fusion breakpoint, a KIF5B primer that hybridizes 5′ to a fusion breakpoint, or a RET primer that hybridizes 5′ to a fusion breakpoint; and wherein an increase in the amount of amplification product relative to a control indicates the presence of an RET or DEPDC1 related cancer.

In one aspect, the disclosed methods utilize in situ hybridization probes. Thus, in one aspect, disclosed herein are methods of diagnosing a RET or DEPDC1 related cancer comprising detecting the presence of or measuring the amount of nucleic acid associated with a nucleic acid variation, over-expression, truncation, or fusions of DEP domain containing 1 gene (DEPDC1) or RET from a tissue sample from the subject; wherein an increase in the amount of amplification product or labeled probe relative to a control indicates the presence of RET or DEPDC1 related cancer, wherein the nucleic acid is measured by microarray or in-situ hybridization method. In one aspect, the disclosed microarray and in-situ hyrbridization methods utilize probes that specifically hybridize to RET or DEPDC1. Thus, in one aspect, disclosed herein are methods of diagnosing an RET or DEPDC1-related cancer, wherein the methods uses one or more probes from the group consisting of SEQ ID NO:5, SEQ ID NO: 7, SEQ ID NO: 11, SEQ ID NO: 14, SEQ ID NO: 17, SEQ ID NO: 20, SEQ ID NO: 23, or SEQ ID NO: 26.

Also disclosed herein are methods of diagnosing a RET-related cancer in a subject with a cancer, comprising obtaining a tissue sample from the subject, isolating nucleic acid from the tissue sample, conducting a nucleic acid amplification process on the nucleic acid, and detecting the presence of or measuring the amount of nucleic acid associated with one or a combination of both wild-type RET and RET kinase domain in the tissue sample, wherein the amplification process is PCR or real-time PCR on cDNA or real-time PCR, RT-PCR, or real-time RT-PCR on mRNA, wherein the PCR, RT-PCR, real-time PCR, or real-time RT-PCR reaction comprises the use of a forward and reverse primer pair that specifically hybridizes to a wild-type RET sequence (such as, for example, primers that bind to the extracellular domain of RET, such as the forward primer SEQ ID NO: 6 and the reverse primer SEQ ID NO: 7) and a forward and reverse primer pair that specifically hybridizes to a wild-type RET kinase domain sequence (such as, for example, the forward primers SEQ ID NOs: 3, 12, or 15) and the reverse primers (SEQ ID NOs: 4, 13, or 16). It is understood that any combination of forward and reverse primer for a particular wild-type RET or RET kinase domain can be used. For example, the forward and reverse primer pair for the wild-type RET (e.g., the ECD of RET) can be SEQ ID NOs: 6 and 7. Similarly, the primers specific for the kinase domain of RET can be any combination of forward and reverse primers for the kinase domain such as, for example SEQ ID NOs: 3 and 4; SEQ ID NOs: 3 and 13; SEQ ID NOs: 3 and 16; SEQ ID NOs: 12 and 4; SEQ ID NOs: 12 and 13; SEQ ID NOs: 12 and 16; SEQ ID NOs: 15 and 4; SEQ ID NOs: 15 and 13; and SEQ ID NOs: 13 and 16. Additionally and combination of ECD RET forward primer and RET kinase reverse primer can be used such as, for example SEQ ID NOs: 6 and 4; SEQ ID NOs: 6 and 13; and SEQ ID NOs: 6 and 16.

Similarly, disclosed herein are methods of diagnosing a DEPDC1-related cancer in a subject with a cancer, comprising obtaining a tissue sample from the subject, isolating nucleic acid from the tissue sample, conducting a nucleic acid amplification process on the nucleic acid, and detecting the presence of or measuring the amount of nucleic acid associated with DEPDC1 in the tissue sample, wherein the amplification process is PCR or real-time PCR on cDNA or real-time PCR, RT-PCR, or real-time RT-PCR on mRNA, wherein the PCR, RT-PCR, real-time PCR, or real-time RT-PCR reaction comprises the use of a forward and reverse primer pair that specifically hybridizes to a DEPDC1 sequence (such as, for example, the forward primers SEQ ID NO: 9, 18, 21, and 24 and the reverse primers SEQ ID NOs: 10, 19, 22, and 25). It is understood that any combination of forward and reverse primer for a particular DEPDC1 can be used. For example, the forward and reverse primer pair for DEPDC1 can be, for example SEQ ID NOs: 9 and 10; SEQ ID NOs: 9 and 19; SEQ ID NOs: 9 and 22; SEQ ID NOs: 9 and 25; SEQ ID NOs: 18 and 10; SEQ ID NOs: 18 and 19; SEQ ID NOs: 18 and 22; SEQ ID NOs: 18 and 25; SEQ ID NOs: 21 and 10; SEQ ID NOs: 21 and 19; SEQ ID NOs: 21 and 22; SEQ ID NOs: 21 and 25; SEQ ID NOs: 24 and 10; SEQ ID NOs: 24 and 19; SEQ ID NOs: 24 and 22; and SEQ ID NOs: 24 and 25.

Fluorescent Change Probes and Primers

Fluorescent change probes and fluorescent change primers refer to all probes and primers that involve a change in fluorescence intensity or wavelength based on a change in the form or conformation of the probe or primer and nucleic acid to be detected, assayed or replicated. Examples of fluorescent change probes and primers include molecular beacons, Amplifluors, FRET probes, cleavable FRET probes, TaqMan probes, scorpion primers, fluorescent triplex oligos including but not limited to triplex molecular beacons or triplex FRET probes, fluorescent water-soluble conjugated polymers, PNA probes and QPNA probes.

Fluorescent change probes and primers can be classified according to their structure and/or function. Fluorescent change probes include hairpin quenched probes, cleavage quenched probes, cleavage activated probes, and fluorescent activated probes. Fluorescent change primers include stem quenched primers and hairpin quenched primers.

Hairpin quenched probes are probes that when not bound to a target sequence form a hairpin structure (and, typically, a loop) that brings a fluorescent label and a quenching moiety into proximity such that fluorescence from the label is quenched. When the probe binds to a target sequence, the stem is disrupted, the quenching moiety is no longer in proximity to the fluorescent label and fluorescence increases. Examples of hairpin quenched probes are molecular beacons, fluorescent triplex oligos, triplex molecular beacons, triplex FRET probes, and QPNA probes.

Cleavage activated probes are probes where fluorescence is increased by cleavage of the probe. Cleavage activated probes can include a fluorescent label and a quenching moiety in proximity such that fluorescence from the label is quenched. When the probe is clipped or digested (typically by the 5′-3′ exonuclease activity of a polymerase during amplification), the quenching moiety is no longer in proximity to the fluorescent label and fluorescence increases. TaqMan probes are an example of cleavage activated probes.

Cleavage quenched probes are probes where fluorescence is decreased or altered by cleavage of the probe. Cleavage quenched probes can include an acceptor fluorescent label and a donor moiety such that, when the acceptor and donor are in proximity, fluorescence resonance energy transfer from the donor to the acceptor causes the acceptor to fluoresce. The probes are thus fluorescent, for example, when hybridized to a target sequence. When the probe is clipped or digested (typically by the 5′-3′ exonuclease activity of a polymerase during amplification), the donor moiety is no longer in proximity to the acceptor fluorescent label and fluorescence from the acceptor decreases. If the donor moiety is itself a fluorescent label, it can release energy as fluorescence (typically at a different wavelength than the fluorescence of the acceptor) when not in proximity to an acceptor. The overall effect would then be a reduction of acceptor fluorescence and an increase in donor fluorescence. Donor fluorescence in the case of cleavage quenched probes is equivalent to fluorescence generated by cleavage activated probes with the acceptor being the quenching moiety and the donor being the fluorescent label. Cleavable FRET (fluorescence resonance energy transfer) probes are an example of cleavage quenched probes.

Fluorescent activated probes are probes or pairs of probes where fluorescence is increased or altered by hybridization of the probe to a target sequence. Fluorescent activated probes can include an acceptor fluorescent label and a donor moiety such that, when the acceptor and donor are in proximity (when the probes are hybridized to a target sequence), fluorescence resonance energy transfer from the donor to the acceptor causes the acceptor to fluoresce. Fluorescent activated probes are typically pairs of probes designed to hybridize to adjacent sequences such that the acceptor and donor are brought into proximity. Fluorescent activated probes can also be single probes containing both a donor and acceptor where, when the probe is not hybridized to a target sequence, the donor and acceptor are not in proximity but where the donor and acceptor are brought into proximity when the probe hybridized to a target sequence. This can be accomplished, for example, by placing the donor and acceptor on opposite ends of the probe and placing target complement sequences at each end of the probe where the target complement sequences are complementary to adjacent sequences in a target sequence. If the donor moiety of a fluorescent activated probe is itself a fluorescent label, it can release energy as fluorescence (typically at a different wavelength than the fluorescence of the acceptor) when not in proximity to an acceptor (that is, when the probes are not hybridized to the target sequence). When the probes hybridize to a target sequence, the overall effect would then be a reduction of donor fluorescence and an increase in acceptor fluorescence. FRET probes are an example of fluorescent activated probes.

Stem quenched primers are primers that when not hybridized to a complementary sequence form a stem structure (either an intramolecular stem structure or an intermolecular stem structure) that brings a fluorescent label and a quenching moiety into proximity such that fluorescence from the label is quenched. When the primer binds to a complementary sequence, the stem is disrupted, the quenching moiety is no longer in proximity to the fluorescent label and fluorescence increases. In the disclosed method, stem quenched primers are used as primers for nucleic acid synthesis and thus become incorporated into the synthesized or amplified nucleic acid. Examples of stem quenched primers are peptide nucleic acid quenched primers and hairpin quenched primers.

Peptide nucleic acid quenched primers are primers associated with a peptide nucleic acid quencher or a peptide nucleic acid fluor to form a stem structure. The primer contains a fluorescent label or a quenching moiety and is associated with either a peptide nucleic acid quencher or a peptide nucleic acid fluor, respectively. This puts the fluorescent label in proximity to the quenching moiety. When the primer is replicated, the peptide nucleic acid is displaced, thus allowing the fluorescent label to produce a fluorescent signal.

Hairpin quenched primers are primers that when not hybridized to a complementary sequence form a hairpin structure (and, typically, a loop) that brings a fluorescent label and a quenching moiety into proximity such that fluorescence from the label is quenched. When the primer binds to a complementary sequence, the stem is disrupted, the quenching moiety is no longer in proximity to the fluorescent label and fluorescence increases. Hairpin quenched primers are typically used as primers for nucleic acid synthesis and thus become incorporated into the synthesized or amplified nucleic acid. Examples of hairpin quenched primers are Amplifluor primers and scorpion primers.

Cleavage activated primers are similar to cleavage activated probes except that they are primers that are incorporated into replicated strands and are then subsequently cleaved.

Labels

To aid in detection and quantitation of nucleic acids produced using the disclosed methods, labels can be directly incorporated into nucleotides and nucleic acids or can be coupled to detection molecules such as probes and primers. As used herein, a label is any molecule that can be associated with a nucleotide or nucleic acid, directly or indirectly, and which results in a measurable, detectable signal, either directly or indirectly. Many such labels for incorporation into nucleotides and nucleic acids or coupling to nucleic acid probes are known to those of skill in the art. Examples of labels suitable for use in the disclosed method are radioactive isotopes, fluorescent molecules, phosphorescent molecules, enzymes, antibodies, and ligands. Fluorescent labels, especially in the context of fluorescent change probes and primers, are useful for real-time detection of amplification.

Examples of suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, CASCADE BLUE®, OREGON GREEN®, pyrene, lissamine, xanthenes, acridines, oxazines, phycoerythrin, macrocyclic chelates of lanthanide ions such as quantum Dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH—CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin E8G, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Phycoerythrin B, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

The absorption and emission maxima, respectively, for some of these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio.

Additional labels of interest include those that provide for signal only when the probe with which they are associated is specifically bound to a target molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037 which is incorporated herein by reference.

Labeled nucleotides are a form of label that can be directly incorporated into the amplification products during synthesis. Examples of labels that can be incorporated into amplified nucleic acids include nucleotide analogs such as BrdUrd, aminoallyldeoxyuridine, 5-methylcytosine, bromouridine, and nucleotides modified with biotin or with suitable haptens such as digoxygenin. Suitable fluorescence-labeled nucleotides are Fluorescein-isothiocyanate-dUTP, Cyanine-3-dUTP and Cyanine-5-dUTP. One example of a nucleotide analog label for DNA is BrdUrd (bromodeoxyuridine, BrdUrd, BrdU, BUdR, Sigma-Aldrich Co). Other examples of nucleotide analogs for incorporation of label into DNA are AA-dUTP (aminoallyl-deoxyuridine triphosphate, Sigma-Aldrich Co.), and 5-methyl-dCTP (Roche Molecular Biochemicals). One example of a nucleotide analog for incorporation of label into RNA is biotin-16-UTP (biotin-16-uridine-5′-triphosphate, Roche Molecular Biochemicals). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Labels that are incorporated into amplified nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo[3.3.1.1^{3,7}]decane]-4-yl) phenyl phosphate; Tropix, Inc.). Labels can also be enzymes, such as alkaline phosphatase, soybean peroxidase, horseradish peroxidase and polymerases, that can be detected, for example, with chemical signal amplification or by using a substrate to the enzyme which produces light (for example, a chemiluminescent 1,2-dioxetane substrate) or fluorescent signal.

Molecules that combine two or more of these labels are also considered labels. Any of the known labels can be used with the disclosed probes, tags, and method to label and detect nucleic acid amplified using the disclosed method. Methods for detecting and measuring signals generated by labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary label coupled to the antibody. As used herein, detection molecules are molecules which interact with amplified nucleic acid and to which one or more labels are coupled.

It is understood and herein contemplated that one method of assessing whether an increase in a particular mRNA or expression of mRNA has occurred or a particular mRNA is present is by comparison with a control sample. Therefore, contemplated herein are methods of diagnosing a cancer in a subject comprising conducting an RT-PCR or PCR reaction with mRNA from a tissue sample from the subject; wherein the reverse transcription polymerase chain reaction (RT-PCR) comprises a reverse primer capable of specifically hybridizing to one or more RET or DEPDC1 sequences and at least one forward primer; wherein an increase in the amount of amplification product relative to a control indicates the presence of an RET or DEPDC1-related cancer; and wherein the control tissue is obtained is a non-cancerous tissue. It is further understood that with respect to RET or DEPDC1-related cancers, the use of a non-cancerous tissue control can be utilized but is not necessary as cancerous tissue from a non-RET or DEPDC1 related cancer may also be used. Thus, disclosed herein are diagnosing RET or DEPDC1 related cancer in a subject comprising conducting an RT-PCR reaction on mRNA from a tissue sample from the subject; wherein the reverse transcription polymerase chain reaction (RT-PCR) comprises a reverse primer capable of specifically hybridizing to one or more RET or DEPDC1 sequences and at least one forward primer; and wherein an increase in the amount of amplification product relative to a control indicates the presence of an RET or DEPDC1 related cancer; and wherein the control tissue is obtained from non-RET or DEPDC1 related cancerous tissue.

The disclosed methods can be used to diagnose any disease where uncontrolled cellular proliferation occurs herein referred to as “cancer”. A non-limiting list of different types of RET or DEPDC1 related cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed methods can be used to diagnose is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

Thus, disclosed herein are methods of diagnosing a cancer wherein the cancer is selected from the group consisting of non-small cell lung carcinoma (including, for example, EGFR, KRAS, and ALK negative NSCLC cancers), diffuse large B-cell lymphoma, systemic histiocytosis, breast cancer, colorectal carcinoma, esophageal squamous cell carcinoma, anaplastic large-cell lymphoma, neuroblastoma, and inflammatory myofibroblastic tumors (IMTs). For example, disclosed herein are methods of diagnosing a RET or DEPDC1-related cancer in a subject comprising conducting an RT-PCR reaction on mRNA from a tissue sample from the subject; wherein the reverse transcription polymerase chain reaction (RT-PCR) comprises a reverse primer capable of specifically hybridizing to one or more RET or DEPDC1 sequences and at least one forward primer; wherein an increase in the amount of amplification product relative to a control indicates the presence of an RET or DEPDC1 related cancer, and wherein the cancer is selected from the group consisting of non-small cell lung carcinoma, diffuse large B-cell lymphoma, systemic histiocytosis, breast cancer, colorectal carcinoma, esophageal squamous cell carcinoma, anaplastic large-cell lymphoma, neuroblastoma, and inflammatory myofibroblastic tumors (IMTs).

### Methods of Assessing the Suitability of RET or DEPDC1 Directed Treatments

Though not wishing to be bound by current theories, it is believed that inhibition of these forms of RET or DEPDC1 genes with small-molecule drug candidates abrogates related abnormal cell proliferation and promotes apoptosis in neuroblastoma and other RET or DEPDC1-related tumor cell lines; furthermore, both preclinical animal models and the early clinical experience with these inhibitors indicate that RET or DEPDC1 small-molecule inhibitors not only possess marked antitumor activity against RET or DEPDC1-related cancers but are also very well tolerated with no limiting target-associated toxicities.

These discoveries highlight the need for a specialized diagnostic test for RET or DEPDC1 mutations. For example, such an assay may be used to screen children in families affected with hereditary neuroblastoma to help facilitate the detection of tumors at an earlier stage when the tumors are more amenable to treatment. Accordingly, disclosed herein are methods of assessing the suitability of a RET or DEPDC1 inhibitor treatment for a cancer in a subject comprising measuring mRNA from a tissue sample from the subject; wherein an increase in the amount of RET or DEPDC1 sequence mRNA relative to a control indicates a cancer that can be treated with an RET or DEPDC1 inhibitor.

It is understood and herein contemplated that any of the disclosed mRNA measuring techniques disclosed herein can be used in these methods. Thus, for example, in one aspect disclosed herein are methods of assessing the susceptibility or risk for a disease or condition, monitoring disease progression, determination of susceptibility or resistance of a cancer to therapeutic RET or DEPDC1 inhibitor treatment for a cancer in a subject or determination of suitability of a RET or DEPDC1 inhibitor treatment for a cancer associated with a nucleic acid variation, truncation, or overexpression of RET or DEPDC1 or RET fusion in a subject comprising conducting an RT-PCR reaction with mRNA or PCR reaction with DNA from a tissue sample from the subject; wherein the RT-PCR reaction comprises a reverse primer capable of specifically hybridizing to one or more RET or DEPDC1 sequences and at least one forward primer; and wherein an increase in the amount of amplification product relative to a control indicates a cancer that can be treated with an RET or DEPDC1 inhibitor. It is further understood that the disclosed methods can further comprise any of the primers disclosed herein and utilize the multiplexing PCR techniques disclosed. Such methods can be accomplished with amplification methods such as PCR, real-time PCR, RT-PCR, or real-time RT-PCR, or by conducting a in situ hybridization methods such as FISH.

In one aspect, disclosed herein are methods for determining the susceptibility or resistance to therapeutic treatment of a cancer to a RET or DEPDC1 inhibitor or suitability of a RET or DEPDC1 inhibitor treatment for a cancer in a subject with a cancer comprising detecting the presence of RET or DEPDC1 kinase activity. Thus, for example, disclosed herein are methods of determining the susceptibility or resistance to therapeutic treatment for an RET or DEPDC1 related cancer or suitability for a cancer to be treated with a RET or DEPDC1 inhibitor in a subject with a cancer comprising obtaining a tissue sample from the subject, isolating nucleic acid from the tissue sample, conducting RT-PCR, real-time PCR, or real-time RT-PCR on the nucleic acid, and detecting the presence of or measuring the amount of nucleic acid associated with RET or DEPDC1 and/or RET kinase domain in the tissue sample, wherein the RT-PCR or real-time PCR reaction comprises the use of a forward and reverse primer pair that specifically hybridizes to a RET or DEPDC1 sequence (e.g., an extracellular domain sequence of RET, such as, SEQ ID NO: 6 and 7) and/or a forward and reverse primer pair that specifically hybridizes to a wild-type RET kinase domain sequence (e.g., SEQ ID SOs: 3, 4, 12, 13, 15 and 16) or a forward and reverse primer pair that specifically hybridizes to a DEPDC1 (e.g., SEQ ID NOs: 9, 10, 18, 19, 21, 22, 24 and 25) and detecting the presence of or measuring the amount of nucleic acid associated with one or a combination of both wild-type RET and RET kinase domain or DEPDC1 in the tissue sample, wherein an increase in amplicon relative to a normal control or the presence of an amplicon indicates the that the subject has a RET or DEPDC1 related cancer and is therefore susceptible to treatment with a RET or DEPDC1 inhibitor. Absence of amplicon or amplicon levels equivalent to normal controls indicates that the cancer is not susceptible to RET or DEPDC1 treatment and would be resistant to such treatment. Also disclosed are methods for determining the susceptibility or resistance to therapeutic treatment for a RET or DEPDC1-related cancer or suitability for a cancer to be treated with a RET or DEPDC1 inhibitor in a subject with a cancer comprising obtaining a tissue sample from the subject, isolating nucleic acid from the tissue sample, wherein the nucleic acid from the tissue sample is RNA, wherein the method further comprises synthesizing cDNA from the RNA sample, conducting PCR on the cDNA; and detecting the presence of or measuring the amount of nucleic acid associated with one or a combination of both wild-type RET and RET kinase domain or DEPDC1 in the tissue sample, wherein an increase in amplicon relative to a normal control or the presence of an amplicon indicates the that the subject has a RET or DEPDC1 related cancer. Where a probe is used, it is understood that the probe can comprise a reporter dye on the end thereof and a quencher dye on the another end thereof. When a cancer is determined to be susceptible to or suitable for treatment with RET or DEPDC1 inhibitors, also disclosed are methods further comprising administering to a subject with a cancer susceptible to RET or DEPDC1 inhibitor treatment, a RET or DEPDC1 inhibitor. Conversely, where the cancer is not susceptible to RET or DEPDC1 inhibitors, the method can further comprise treating the subject with the cancer using a form of treatment other than a RET or DEPDC1 inhibitor.

### Methods of Screening

The RET or DEPDC1-fusions, over-expression and point mutations disclosed herein are targets for cancer treatments. Thus, disclosed herein are method of screening for an agent that inhibits an RET or DEPDC1 related cancer in a subject comprising

a) obtaining a tissue sample from a subject with an RET or DEPDC1 related cancer;

b) contacting the tissue sample with the agent

c) extracting mRNA from the tissue sample;

d) conducting an RT-PCR reaction on the mRNA from the tissue sample;

wherein the RT-PCR reaction comprises a reverse primer capable of specifically hybridizing to one or more RET or DEPDC1 sequences and at least one forward primer; and wherein a decrease in the amount of amplification product relative to an untreated control indicates an agent that can inhibit an RET or DEPDC1 related cancer.

### Nucleic Acids

The disclosed method and compositions make use of various nucleic acids.

Generally, any nucleic acid can be used in the disclosed method. For example, the disclosed nucleic acids of interest and the disclosed reference nucleic acids can be chosen based on the desired analysis and information that is to be obtained or assessed. The disclosed methods also produce new and altered nucleic acids. The nature and structure of such nucleic acids will be established by the manner in which they are produced and manipulated in the methods. Thus, for example, extension products and hybridizing nucleic acids are produced in the disclosed methods. As used herein, hybridizing nucleic acids are hybrids of extension products and the second nucleic acid.

It is understood and contemplated herein that a nucleic acid of interest can be any nucleic acid to which the determination of the presence or absence of nucleotide variation is desired. Thus, for example, the nucleic acid of interest can comprise a sequence that corresponds to the wild-type sequence of the reference nucleic acid. It is further disclosed herein that the disclosed methods can be performed where the first nucleic acid is a reference nucleic acid and the second nucleic acid is a nucleic acid of interest or where the first nucleic acid is the nucleic acid of interest and the second nucleic acid is the reference nucleic acid.

It is understood and herein contemplated that a reference nucleic acid can be any nucleic acid against which a nucleic acid of interest is to be compared. Typically, the reference nucleic acid has a known sequence (and/or is known to have a sequence of interest as a reference). Although not required, it is useful if the reference sequence has a known or suspected close relationship to the nucleic acid of interest. For example, if a single nucleotide variation is desired to be detected, the reference sequence can be usefully chosen to be a sequence that is a homolog or close match to the nucleic acid of interest, such as a nucleic acid derived from the same gene or genetic element from the same or a related organism or individual. Thus, for example, it is contemplated herein that the reference nucleic acid can comprise a wild-type sequence or alternatively can comprise a known mutation including, for example, a mutation the presence or absence of which is associated with a disease or resistance to therapeutic treatment. Thus, for example, it is contemplated that the disclosed methods can be used to detect or diagnose the presence of known mutations in a nucleic acid of interest by comparing the nucleic acid of interest to a reference nucleic acid that comprises a wild-type sequence (i.e., is known not to possess the mutation) and examining for the presence or absence of variation in the nucleic acid of interest, where the absence of variation would indicate the absence of a mutation. Alternatively, the reference nucleic acid can possess a known mutation. Thus, for example, it is contemplated that the disclosed methods can be used to detect susceptibility for a disease or condition by comparing the nucleic acid of interest to a reference nucleic acid comprising a known mutation that indicates susceptibility for a disease and examining for the presence or absence of the mutation, wherein the presence of the mutation indicates a disease.

Herein, the term “nucleotide variation” refers to any change or difference in the nucleotide sequence of a nucleic acid of interest relative to the nucleotide sequence of a reference nucleic acid. Thus, a nucleotide variation is said to occur when the sequences between the reference nucleic acid and the nucleic acid of interest (or its complement, as appropriate in context) differ. Thus, for example, a substitution of an adenine (A) to a guanine (G) at a particular position in a nucleic acid would be a nucleotide variation provided the reference nucleic acid comprised an A at the corresponding position. It is understood and herein contemplated that the determination of a variation is based upon the reference nucleic acid and does not indicate whether or not a sequence is wild-type. Thus, for example, when a nucleic acid with a known mutation is used as the reference nucleic acid, a nucleic acid not possessing the mutation (including a wild-type nucleic acid) would be considered to possess a nucleotide variation (relative to the reference nucleic acid).

### Nucleotides

The disclosed methods and compositions make use of various nucleotides. Throughout this application and the methods disclosed herein reference is made to the type of base for a nucleotide. It is understood and contemplated herein that where reference is made to a type of base, this refers a base that in a nucleotide in a nucleic acid strand is capable of hybridizing (binding) to a defined set of one or more of the canonical bases. Thus, for example, where reference is made to extension products extended in the presence of three types of nuclease resistant nucleotides and not in the presence of nucleotides that comprise the same type of base as the modified nucleotides, this means that if, for example, the base of the modified nucleotide was an adenine (A), the nuclease-resistant nucleotides can be, for example, guanine (G), thymine (T), and cytosine (C). Each of these bases (which represent the four canonical bases) is capable of hybridizing to a different one of the four canonical bases and thus each qualify as a different type of base as defined herein. As another example, inosine base pairs primarily with adenine and cytosine (in DNA) and thus can be considered a different type of base from adenine and from cytosine-which base pair with thymine and guanine, respectively—but not a different type of base from guanine or thymine-which base pair with cytosine and adenine, respectively-because the base pairing of guanine and thymine overlaps (that is, is not different from) the hybridization pattern of inosine

Any type of modified or alternative base can be used in the disclosed methods and compositions, generally limited only by the capabilities of the enzymes used to use such bases. Many modified and alternative nucleotides and bases are known, some of which are described below and elsewhere herein. The type of base that such modified and alternative bases represent can be determined by the pattern of base pairing for that base as described herein. Thus for example, if the modified nucleotide was adenine, any analog, derivative, modified, or variant base that based pairs primarily with thymine would be considered the same type of base as adenine. In other words, so long as the analog, derivative, modified, or variant has the same pattern of base pairing as another base, it can be considered the same type of base. Modifications can made to the sugar or phosphate groups of a nucleotide. Generally such modifications will not change the base pairing pattern of the base. However, the base pairing pattern of a nucleotide in a nucleic acid strand determines the type of base of the base in the nucleotide.

Modified nucleotides to be incorporated into extension products and to be selectively removed by the disclosed agents in the disclosed methods can be any modified nucleotide that functions as needed in the disclosed method as is described elsewhere herein. Modified nucleotides can also be produced in existing nucleic acid strands, such as extension products, by, for example, chemical modification, enzymatic modification, or a combination.

Many types of nuclease-resistant nucleotides are known and can be used in the disclosed methods. For example, nucleotides have modified phosphate groups and/or modified sugar groups can be resistant to one or more nucleases. Nuclease-resistance is defined herein as resistance to removal from a nucleic acid by any one or more nucleases. Generally, nuclease resistance of a particular nucleotide can be defined in terms of a relevant nuclease. Thus, for example, if a particular nuclease is used in the disclosed method, the nuclease-resistant nucleotides need only be resistant to that particular nuclease. Examples of useful nuclease-resistant nucleotides include thio-modified nucleotides and borano-modified nucleotides.

There are a variety of molecules disclosed herein that are nucleic acid based. Non-limiting examples of these and other molecules are discussed herein. It is understood that for example, a nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (adenine, A), cytosin-1-yl (cytosine, C), guanin-9-yl (guanine, G), uracil-1-yl (uracil, U), and thymin-1-yl (thymine, T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl (ψ), hypoxanthin-9-yl (inosine, I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, which is incorporated herein in its entirety for its teachings of base modifications. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Often time base modifications can be combined with for example a sugar modification, such as 2′-O-methoxyethyl, to achieve unique properties such as increased duplex stability.

Nucleotide analogs can also include modifications of the sugar moiety. Modifications to the sugar moiety would include natural modifications of the ribose and deoxy ribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10, alkyl or C2 to C10 alkenyl and alkynyl. 2′ sugar modifications also include but are not limited to —O[(CH2)n O]m CH3, —O(CH2)n OCH3, —O(CH2)n NH2, —O(CH2)n CH3, —O(CH2)n —ONH2, and —O(CH2)nON[(CH2)n CH3)]2, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH3, OCN, Cl, Br, CN, CF3, OCF3, SOCH3, SO2 CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH2 and S. Nucleotide sugar analogs may also have sugar mimetics such as cyclobutyl moieties in place of the pentofuranosyl sugar.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included.

It is understood that nucleotide analogs need only contain a single modification, but may also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference.

It is also possible to link other types of molecules (conjugates) to nucleotides or nucleotide analogs. Conjugates can be chemically linked to the nucleotide or nucleotide analogs. Such conjugates include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

### Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### Kits

Disclosed herein are kits that are drawn to reagents that can be used in practicing the methods disclosed herein. In particular, he kits can include any reagent or combination of reagents discussed herein or that would be understood to be required or beneficial in the practice of the disclosed methods. For example, the kits could include one or more primers disclosed herein to perform the extension, replication and amplification reactions discussed in certain embodiments of the methods, as well as the buffers and enzymes required to use the primers as intended.

It is understood that to detect an RET related fusion or RET or DEPDC1 overexpression, a reverse primer can be used that hybridizes with wild-type RET or DEPDC1 or a RET kinase domain. Thus, disclosed herein are kits that include at least one reverse primer wherein the reverse primer hybridizes to a portion of wild-type RET or DEPDC1 such as the kinase domain of RET, a RET extracellular domain, or DEPDC1 3′ of any fusion breakpoint. Examples of reverse primers that can be used in the disclosed kits include but are not limited to SEQ ID NO. 4, SEQ ID NO: 7, SEQ ID NO: 10, SEQ ID NO: 13, SEQ ID NO: 16, SEQ ID NO: 19, SEQ ID NO: 22, and SEQ ID NO: 25. Additionally, it is understood that the kits disclosed herein can include one or more forward primers that specifically hybridize to a fusion partner of RET or wild-type RET, a RET kinase, or DEPDC1. Thus, for example the forward primer can hybridize to wild-type RET or 5′ of a fusion breakpoint, KIF5B 5′ of a fusion breakpoint, or CCDC6 5′ of a fusion breakpoint, or NCOA4 5′ of a fusion breakpoint or DEPDC1 (where a DEPDC1 reverse primer is used). A non-limiting list of forward primers that can be used in the kits disclosed herein include but are not limited to SEQ ID NO: 3, SEQ ID NO: 6, SEQ ID NO: 9, SEQ ID NO: 12, SEQ ID NO: 15, SEQ ID NO: 18, SEQ ID NO: 21, and SEQ ID NO: 24. One of skill in the art can appreciate that it is suitable to have a kit that comprises more than a singular primer pair and could include, for example, a single reverse primer, such as SEQ ID NO: 4, SEQ ID NO: 13, and/or SEQ ID NO: 16, and multiple forward primers. Thus, specifically contemplated herein are kits for RET related cancers including one or more forward primers that specifically hybridizes to wild-type RET, KIF5B, NCOA4, or CCDC6 and at least one reverse primer, wherein the reverse primer is a wild-type RET reverse primer such as SEQ ID NOs. 4, 13, or 16). In one aspect, disclosed herein are kits wherein the forward primer binds to wild-type RET 5′ of any fusion break point (e.g., SEQ ID NO: 6). In another aspect, disclosed herein are kits wherein the forward primer specifically hybridizes to RET kinase (e.g. SEQ ID NOS: 3, 12, and 15). Also disclosed are kits wherein the forward primer hybridizes to DEPDC1 (e.g., SEQ ID NOs: 9, 18, 21, and 24). Also disclosed are kits comprising any of the probes disclosed herein such as, for example, the RET binding probes SEQ ID NO: 5, SEQ ID NO: 8, SEQ ID NO: 14. and/or SEQ ID NO: 17) and/or the DPEDC1 hybridizing probes SEQ ID NO: 11, SEQ ID NO: 20, SEQ ID NO: 23, and/or SEQ ID NO: 26.

It is understood that the disclosed kits can also include controls to insure the methods disclosed herein are properly functioning and to normalize results between assays. Thus, for example, disclosed herein are positive cDNA controls, negative cDNA controls, and control primer pairs. For example, the disclosed kits can include a control primer pairs for the detection of Homo sapiens ATP synthase, H+ transporting, mitochondrial F1 complex, O subunit (ATP5O), nuclear gene encoding mitochondrial protein mRNA; Homo sapiens NADH dehydrogenase (ubiquinone) 1 alpha subcomplex, 2, 8 kDa (NDUFA2), mRNA; Homo sapiens glyceraldehyde-3-phosphate dehydrogenase (GAPDH), mRNA; Homo sapiens H3 histone, family 3A (H3F3A), mRNA; Homo sapiens proteasome (prosome, macropain) subunit, beta type, 4 (PSMB4), mRNA; Homo sapiens ribosomal protein S27a (RPS27A), transcript variant 1, mRNA; Homo sapiens eukaryotic translation initiation factor 4A, isoform 2 (EIF4A2), mRNA; Homo sapiens ribosomal protein L18 (RPL18), mRNA; Homo sapiens adenosine deaminase, RNA-specific (ADAR), transcript variant 1, mRNA; or Homo sapiens cytochrome c oxidase subunit Vb (COX5B), mRNA. Examples of primers pairs include but are not limited to the primer pairs found in Table 1.

TABLE 1

| Sequence Name | Sense Primer | Anti-Sense Primer |
|---|---|---|
| ATP5O | GCGTTTCTCTCTTCCCACTC | GGCATAGCGACCTTCAATACC |
| | (SEQ ID NO: 27 | (SEQ ID NO: 37) |
| NDUFA | GCCTGAAGACCTGGAATTGG | CTGACATAAGTGGATGCGAATC |
| | (SEQ ID NO: 28) | (SEQ ID NO: 38) |
| GAPDH | GGAAGGTGAAGGTCGGAGTC | GCTGATGATCTTGAGGCTGTTG |
| | (SEQ ID NO: 29) | (SEQ ID NO: 39) |
| H3F3A | CCAGCCGAAGGAGAAGGG | AGGGAAGTTTGCGAATCAGAAG |
| | (SEQ ID NO: 30) | (SEQ ID NO: 40) |
| PSMB4 | TACCGCATTCCGTCCACTC | GCTCCTCATCAATCACCATCTG |
| | (SEQ ID NO: 31) | (SEQ ID NO: 41) |
| RPS27A | CGGCAGTCAGGCATTTGG | CCACCACGAAGTCTCAACAC |
| | (SEQ ID NO: 32) | (SEQ ID NO: 42) |
| EIF4A2 | CTCTCCTTCGTGGCATCTATG | GGTCTCCTTGAACTCAATCTCC |
| | (SEQ ID NO: 33) | (SEQ ID NO: 43) |
| RPL18 | GGACATCCGCCATAACAAGG | ACAACCTCTTCAACACAACCTG |
| | (SEQ ID NO: 34) | (SEQ ID NO: 44) |
| ADAR | AGACGGTCATAGCCAAGGAG | GCAGAGGAGTCAGACACATTG |
| | (SEQ ID NO: 35) | (SEQ ID NO: 45) |
| COX5B | ACGCAATGGCTTCAAGGTTAC | CGCTGGTATTGTCCTCTTCAC |
| | (SEQ ID NO: 36) | (SEQ ID NO: 46) |

Additionally, it is understood that the disclosed kits can include such other reagents and material for performing the disclosed methods such as a enzymes (e.g., polymerases), buffers, sterile water, reaction tubes. Additionally the kits can also include modified nucleotides, nuclease-resistant nucleotides, and or labeled nucleotides. Additionally, the disclosed kits can include instructions for performing the methods disclosed herein and software for enable the calculation of the presence of a RET or DEPDC1 mutation.

In one aspect, the disclosed kits can comprise sufficient material in a single assay run simultaneously or separately to conduct the methods to determine if a sample contains a wild-type RET or DEPDC1, a known RET or DEPDC1 fusion, or a previously unidentified RET or DEPDC1 fusion. The kits can also include sufficient material to run control reactions. Thus, disclosed herein, in one aspect, are kits comprising a positive cDNA control reaction tube, a negative cDNA control reaction tube, a control primer reaction tube, and one or more reaction tubes to detect known ALK fusions, wild-type ALK, and/or RET or DEPDC1 kinase activity.

### Nucleic Acid Synthesis

The disclosed nucleic acids, such as, the oligonucleotides to be used as primers can be made using standard chemical synthesis methods or can be produced using enzymatic methods or any other known method. Such methods can range from standard enzymatic digestion followed by nucleotide fragment isolation to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B).

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### KIF5B-RET Therapy

Small-molecule tyrosine kinase inhibitors (TKI) have been used extensively for years to target and inhibit various RET or DEPDC1 but to date none exist exclusively for targeting RET. Using ambiguous TKIs such as Vandetanib (ZD6474), Sorafenib (BAY 43-9006), Sunitinib and XL-184 have shown significant activity against RET indicating these TKIs could be FDA approved for use in RET positive patients similar to the case of the multikinase first-in-class ALK inhibitor XALKORI® (crizotinib, Pfizer). XALKORI® is a small-molecule inhibitor shown originally to inhibit the catalytic activity of the c-Met kinase and latter shown to have similar activity against the kinase domain of ALK fusions. XALKORI® appears to be selective for both c-Met and ALK at pharmacologically relevant concentrations demonstrating excellent antitumor responses and a lack of any significant toxicities. Demonstrating multifaceted inhibitory activity from a single TKI against various dysregulated RET or DEPDC1s gives indicates that targeted therapy can be made available to patients with multiple oncogenic drivers while minimizing the side effects of large drug regimens.

### Example 2

### DEPDC1 Therapy

The expression of DEPDC1 has been associated with triple negative lung cancer patients in a specific subgroup with poor prognosis supporting the use of therapeutics targeting DEPDC1. Phase I/II studies using novel epitope peptides derived against DEPDC1 have proven tolerable and efficacious for patients with bladder cancer. The peptide trials have been extended into vaccine trials to prevent recurrence and are ongoing. Diagnostics targeting biomarkers such as DEPDC1 are contemporaneous development to achieve full FDA approval of theses therapeutics in triple negative NSCLC cases.

### Example 3

### Development of a PCR-Based Assay to Detect KIF5B-RET Fusions

The KIF5B-RET PCR diagnostic as disclosed herein can be performed as a one-step real-time RT-PCR test. This method was developed to identify all RET fusions regardless of the fusion partner or DEPDC1 or RET overexpression. The underlying design for the screen is illustrated in FIG. 2A and shows that, in the case of RET, the assay targets the oncogenic expression of the RET kinase which is typically not expressed in lung tissue. Therefore, detection of RET kinase expression is indicative of an oncogenic RET fusion. For RET, the assay utilizes a single PCR primer set that amplifies a RET gene segment encoding the kinase domain of RET. Disclosed in Table 2. are compositions including primers and probes for the assay. The qPCR design amplifies a RET gene segment encoding the intracellular kinase domain, which is found both in normal and fused RET forms allowing the assay design to detect the presence of any RET fusion and also over-expression of the intact RET gene. Similar to ALK, RET activation is driven by chromosomal translocations, inversions and specific point mutations all of which are detected by the proposed qPCR design. As noted, this elegant and simple design identifies both known and unknown RET fusions independent of the fusion partner involved; in addition, it identifies and quantifies over-expression of wild-type RET. All RET fusions known or unknown along with full length RET mRNA expression is correlated with internal control standards.

TABLE 2
Oligonucleotide specifications for each primer & probe used in the Insight ROS1
Screen version 2 ™

| Gene | Primer/Probe | Sequence (5′-3′) |
|---|---|---|
| KIF5B-RET Kinase domain | Forward Primer | TGGCCGTGAAGATGCTGAAAGAGA |
| | | (SEQ ID NO: 3) |
| KIF5B-RET Kinase domain | Reverse Primer | TGATGACATGTGGGTGGTTGACCT |
| | | (SEQ ID NO: 4) |
| KIF5B-RET Kinase domain | Probe | /56-FAM/ACCTGCTGT/ZEN/CAGAGTTCAACGTCCTGAA/3IABk |
| | | (SEQ ID NO: 5) |
| RET Wild Type | Forward Primer | CCAGTACCTACTCCCTCTCCGTGA |
| | | (SEQ ID NO: 6) |
| RET Wild Type | Reverse Primer | TGTACTGGACGTTGATGCCACTGA |
| | | (SEQ ID NO: 7) |
| RET Wild Type | Probe | /56-FAM/TTTGCCCAG/ZEN/ATCGGGAAAGTCTGTGT/3IABk |
| | | (SEQ ID NO: 8) |
| DEPDC1 | Forward Primer | TGCAATGGGTACGAGGTCACTGAT |
| | | (SEQ ID NO: 9) |
| DEPDC1 | Reverse Primer | CCAGCAAGAAGCTCATCAAGATCC |
| | | (SEQ ID NO: 10) |
| DEPDC1 | Probe | /56-FAM/CTCGATGTG/ZEN/TGTTATGCTGTGCTGAAGA/3IABkF |
| | | (SEQ ID NO: 11) |
| RET Kinase domain | Forward Primer | GGAGAAGGCGAATTTGGAAAAG |
| | | (SEQ ID NO: 12) |
| RET Kinase domain | Reverse Primer | CAGGACGTTGAACTCTGACAG |
| | | (SEQ ID NO: 13) |
| RET Kinase domain | Probe | TGGCCGTGAAGATGCTGAAAGAGA |
| | | (SEQ ID NO: 14) |
| RET Kinase domain | Forward Primer | ATCTGAAAGGCAGAGCAGGGTACA |
| | | (SEQ ID NO: 15) |
| RET Kinase domain | Reverse Primer | TGACCTGCTTCAGGACGTTGAACT |
| | | (SEQ ID NO: 16) |
| Ret Kinase domain | Probe | GAAAGAGAACGCCTCCCCGAGTGAGCTT |
| | | (SEQ ID NO: 17) |
| DEPDC1 | Forward Primer | AAGACTCTGCAAAAGTACAATAGAAC |
| | | (SEQ ID NO: 18) |
| DEPDC1 | Reverse Primer | CGATGGCAACCCTCTCTAAATG |
| | | (SEQ ID NO: 19) |
| DEPDC1 | Probe | AGCAAGCTTTGTGTGCCAGTCAAC |
| | | (SEQ ID NO: 20) |
| DEPDC1 | Forward Primer | ATCTCCCTGAACCTCTACTTACT |
| | | (SEQ ID NO: 21) |
| DEPDC1 | Reverse Primer | AACTGTAGAGCATCGATGGCAA |
| | | (SEQ ID NO: 22) |
| DEPDC1 | Probe | ACATTTTGGGCTTGCTGCAACCTCATTTAGAGAG |
| | | (SEQ ID NO: 23) |
| DEPDC1 | Forward Primer | TGCAATGGGTACGAGGTCACTGAT |
| | | (SEQ ID NO: 24) |
| DEPDC1 | Reverse Primer | CCAGCAAGAAGCTCATCAAGATCC |
| | | (SEQ ID NO: 25) |
| DEPDC1 | Probe | CTCGATGTGTGTTATGCTGTGCTGAAGA |
| | | (SEQ ID NO: 26) |

### Example 4

### Development of a PCR-Based Assay to Detect DEPDC1

The DEPDC1 PCR diagnostic is a real-time PCR or real-time RT-PCR test. The underlying design for Insight DEPDC1 Screen is illustrated in FIG. 2B. The assay utilizes a single PCR primer set that amplifies a DEPDC1 gene segment which identifies and quantifies over-expression of wild-type DEPDC1. Disclosed in Table 2. are compositions including primers and probes for the assay. DEPDC1 mRNA expression is correlated with internal control standards.

### Example 5

### Optimization of RT-PCR Primers

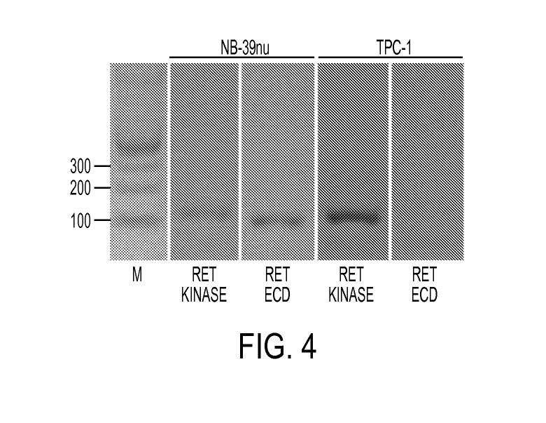

Primers for RT-PCR have been optimized to a binding Tm of 60° C. for the generation of putative target DNA. These primers have been optimized as single amplicon reactions; however, several or all can be batched to allow multiplexing of putative target DNA. FIG. 4 shows gel electrophoresis of target DNAs amplified from each RET or DEPDC1 variant, over-expressor, and fusion of interest. PCR amplification protocol utilized 35 cycles with 95° C. for 15 min; 94° C. for 30 s; 52° C. for 1 min; 72° C. for 1 min; 72° C. for 15 min, and 4° C.

### Example 6

### Insight RET Screen Development

The Insight RET Screen was optimized for performance using a thyroid carcinoma cell line, TPC-1, which expresses the RET receptor tyrosine kinase (TK) proto-oncogene (RET/PTC) specific for thyroid cancer and detectable by the Insight RET Screen. The limited number of available cell lines expressing aberrant RET expression made it prudent to identify other cell lines which express full length RET for control purposes. Although not the primary oncogenic driver, neuroblastoma cell lines express the full-length RET receptor. These cell lines were therefore used as an additional positive control in development studies. To address the specificity of the Insight RET Screen, a series of lung cancer-derived cell lines were tested which express various oncogenic RTK fusions (Table 3). The RET fusion-positive cell line was detected with a ΔCt of 3.71, while the two ALK-positive cell lines were detected at ΔCt greater than 11. The ALK-positive cell lines were reflexed screened by Sanger sequencing and confirmed to express full-length RET. This observation emphasizes the sensitivity of the assay, but the clinical significance of full-length RET expression observed in cell lines has yet to be determined.

TABLE 3
Specificity of Insight RET Screen.

| Template | Mutations | Insight RET Screen | Control | ΔCt |
|---|---|---|---|---|
| TPC-1 | RET/PTC | 24.53 ± 0.15 | 20.82 ± 1.41 | 3.71 |
| H2228 | ALK fusions | 34.65 ± 0.45 | 20.29 ± 0.83 | 14.36 |
| H3122 | ALK fusions | 32.79 ± 0.73 | 21.19 ± 0.97 | 11.6 |
| HCC78 | ROS1 fusion | ND | 21.64 ± 1.08 | ND |
| A549 | KRAS mutations | 33.49 ± 0.15 | 21.97 ± 1.08 | 11.52 |

Total RNA from a cell line expressing a RET fusion (TPC-1), ALK fusions (H2228, H3122), ROS1 fusions (HCC78), and KRAS mutations (A549) was used as template for one-step PCR amplification.

In order to develop a control for the detection of full-length RET expression, a companion extracellular domain (ECD)-specific target assay was developed. This control reaction was used in tandem with the Insight RET Screen to differentiate RET full length versus fusion expression in TPC-1 cells compared to full-length expression of RET in NB-39nu neuroblastoma-derived fusion negative cells (FIG. 4). The results of this study demonstrate the specificity of the ECD assay and indicate the ΔCts observed in the TPC-1 cell lines were due to strict RET fusion expression and complete absence of full length RET. To test the performance of the Insight RET Screen in cell lines, total RNA from the RET fusion-expressing cell line, TPC-1, was diluted to extinction and used as template for the assay (Table 4). Results from this study indicate a limit of detection of ing of total RNA. Total RNA from TPC-1 was then diluted to extinction in a constant amount of negative (Jurkat cell line) control RNA (Table 5). Results from this study indicate that RET fusion transcripts can be detected in a mixture of 1 RET fusion cell line in the background of 10,000 RET negative cell lines (1:10,000). Such high levels of sensitivity should be sufficiently stringent to identify even rare driver RET mutations in a high level of heterogeneous normal tissue.

TABLE 4
Limit of Detection of Insight RET Screen.

| Template | Insight RET Screen | Control | ΔCt |
|---|---|---|---|
| TPC-1 50 ng | 24.58 ± 0.12 | 24.43 ± 0.09 | 0.15 |
| TPC-1 10 ng | 29.12 ± 0.35 | 29.15 ± 0.30 | −0.03 |
| TPC-1 1 ng | 33.79 ± 1.71 | 32.46 ± 0.07 | 1.33 |
| TPC-1 0.1 ng | ND | 36.36 ± 0.98 | ND |
| TPC-1 0.01 ng | ND | ND | ND |

Total RNA from a cell line expressing a RET fusion (TPC-1) was used at limiting input for one-step PCR amplification.

TABLE 5
Sensitivity of Insight RET Screen for the RET Fusion.

| Template | Insight RET Screen | Control | ΔCt |
|---|---|---|---|
| TPC-1 | 30.03 ± 0.65 | 26.24 ± 0.63 | 3.8 |
| TPC-1/WT 1:1 | 30.14 ± 1.57 | 24.79 ± 2.24 | 5.34 |
| TPC-1/WT 1:10 | 31.70 ± 1.88 | 22.82 ± 1.27 | 8.87 |
| TPC-1/WT 1:100 | 35.54 ± 1.29 | 23.14 ± 0.23 | 12.4 |
| TPC-1/WT 1:1000 | 36.52 ± 0.00 | 22.53 ± 1.13 | 13.99 |
| TPC-1/WT 1:10000 | 38.43 ± 0.19 | 23.47 ± 1.18 | 14.96 |
| Wild-Type | ND | 23.82 ± 0.71 | ND |

Total RNA from a cell line expressing a RET fusion (TPC-1) was diluted into constant wild-type RET-negative total RNA as input for one-step PCR amplification.

### Example 7

### Pre-Clinical Validation of the Insight RET Screen

The target specimen for the Insight RET Screen is formalin-fixed paraffin-embedded (FFPE) tissue from lung cancer biopsies. To demonstrate that the in vitro assay performance is suitable for clinical specimen testing, a series of FFPE specimens derived from lung adenocarcinoma biopsies were screened using the Insight RET Screen. Observations from various groups indicate an incidence rate of 1.3% in NSCLC (Pao and Hutchinson Nat Medicine 2012). A total of 92 FFPE specimens were tested with the Insight RET Screen to evaluate performance of the assay. The 92 specimens were divided into two groups: blinded lung adenocarcinomas (LA) and pre-screened EGFR- and KRAS-negative (double-negative, DN) adenocarcinomas which is enriched for RET fusion-positive specimens. A total of 21 specimens were detected in unknown and double-negative adenocarcinomas by the Insight RET Screen with a 20% and 29% call rate, respectively. Evaluation of the detected specimens by a RT-PCR-based Sanger sequencing confirmed a total of 9 specimens (9.8%) with an average ΔCt of 6.88±1.4 (LA 6.97, DN 6.77). The remaining specimens which were detected by Insight RET Screen, but were not detected by Sanger sequencing had an average ΔCt of 12.68±1.4 (LA 13.44, DN 11.18).

TABLE 6
Identification of RET-positive FFPE
Specimens by Insight RET Screen.

| Type | Specimens | Detected | Confirmed | Avg ΔCt |
|---|---|---|---|---|
| Lung adenocarcinoma | 64 | 13 (20%) | 4 (6.3%) | 6.97 ± 1.51 |
| EGFR-, KRAS- | 28 | 8 (29%) | 5 (18%) | 6.77 ± 1.37 |
| negative | | | | |

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

### REFERENCES

Alberti L, Carniti C, Miranda C, Roccato E, Pierotti M A. RET and NTRK1 proto-oncogenes in human diseases. J Cell Physiol. 2003 May; 195(2):168-86.

Attié-Bitach T, Abitbol M, Gérard M, Delezoide A L, Augé J, Pelet A, Amiel J, Pachnis V, Munnich A, Lyonnet S, Vekemans M. Expression of the RET proto-oncogene in human embryos. Am J Med Genet. 1998 Dec. 28; 80(5):481-6.
Carlomagno F, Anaganti S, Guida T, et al. BAY 43-9006 inhibition of oncogenic RET mutants. J Natl Cancer Inst 2006; 98:326-34.
Carlomagno F, Guida T, Anaganti S, et al. Disease associated mutations at valine 804 in the RET receptor tyrosine kinase confer resistance to selective kinase inhibitors. Oncogene 2004; 23:6056-63.
Carlomagno F, Guida T, Anaganti S, et al. Identification of tyrosine 806 as a molecular determinant of RET kinase sensitivity to ZD6474. Endocr Relat Cancer 2009; 16:233-41.
Carlomagno F, Vitagliano D, Guida T, et al. ZD6474, an orally available inhibitor of KDR tyrosine kinase activity, efficiently blocks oncogenic RET kinases. Cancer Res 2002; 62:7284-90.
Christensen J G, Zou H Y, Arango M E, et al. Cytoreductive antitumor activity of PF-2341066, a novel inhibitor of anaplastic lymphoma kinase and c-Met, in experimental models of anaplastic large-cell lymphoma. Mol Cancer Ther 2007; 6:3314-22.
Dawson D M, Lawrence E G, MacLennan G T, Amini S B, Kung H J, Robinson D, Resnick M I, Kursh E D, Pretlow T P, Pretlow T G. Altered expression of RET proto-oncogene product in prostatic intraepithelial neoplasia and prostate cancer. J Natl Cancer Inst. 1998 Apr. 1; 90(7):519-23.
De Souza J A, Busaidy N, Zimrin A, et al. Phase II trial of sunitinib inmedullary thyroid cancer (MTC). J Clin Oncol 2010; 28:5504.
Eisen M B, Spellman P T, Brown P O, Botstein D. Cluster analysis and display of genome-wide expression patterns. Proc Natl Acad Sci USA. 1998 Dec. 8; 95(25):14863-8.
Ferlay J, Shin HR, Bray F, Forman D, Mathers C, Parkin D M. Estimates of worldwide burden of cancer in 2008: GLOBOCAN 2008. Int J Cancer. 2010 Dec. 15; 127(12):2893-91.
Flavin R, Finn S P, Choueiri T K, et al.: RET protein expression in papillary renal cell carcinoma. Urol Oncol 30:900-905, 2012
Futami H, Sakai R: RET protein promotes non-adherent growth of NB-39-nu neuroblastoma cell line. Cancer Sci 100:1034-1039, 2009
Gramza A, Patterson J, Peters J, et al.: Activity of novel RET inhibitors against RET genotypes associated with medullary thyroid cancer. J Clin Oncol 28:5559, 2010
Harada Y, Kanehira M, Fujisawa Y, Takata R, Shuin T, Miki T, Fujioka T, Nakamura Y, Katagiri T. Cell-permeable peptide DEPDC1-ZNF224 interferes with transcriptional repression and oncogenicity in bladder cancer cells. Cancer Res. 2010 Jul. 15; 70(14):5829-39. Epub 2010 Jun. 29.
Ju Y S, Lee W C, Shin J Y, Lee S, Bleazard T, Won J K, Kim Y T, Kim J I, Kang J H, Seo J S. A transforming KIF5B and RET gene fusion in lung adenocarcinoma revealed from whole-genome and transcriptome sequencing. Genome Res. 2012 Jan. 23. [Epub ahead of print].
Kanehira M, Harada Y, Takata R, Shuin T, Miki T, Fujioka T, Nakamura Y, Katagiri T. Involvement of upregulation of DEPDC1 (DEP domain containing 1) in bladder carcinogenesis. Oncogene. 2007 Sep. 27; 26(44):6448-55. Epub 2007 Apr. 23.
Klugbauer S, Jauch A, Lengfelder E, Demidchik E, Rabes H M. A novel type of RET rearrangement (PTC8) in childhood papillary thyroid carcinomas and characterization of the involved gene (RFG8). Cancer Res. 2000 Dec. 15; 60(24):7028-32.
Kohno T, Ichikawa H, Totoki Y, et al.: KIF5B-RET fusions in lung adenocarcinoma. Nat Med 18:375-377, 2012
Kretschmer C, Sterner-Kock A, Siedentopf F, Schoenegg W, Schlag P M, Kemmner W. Identification of early molecular markers for breast cancer. Mol Cancer. 2011 Feb. 11; 10(1):15.
Kurzrock R, Cohen E E, Sherman S I, et al. Long-term results in a cohort of medullary thyroid cancer (MTC) patients (pts) in a phase I study of XL184 (BMS 907351), an oral inhibitor of MET, VEGFR2, and RET. J Clin Oncol 2010; 28:5502.
Kurzrock R, Sherman S, Hong D S, et al. A phase I study of XL184, aMET, VEGFR2, and RET kinase inhibitor, administered orally to patients with advanced malignancies, including a subgroup of patients with medullary thyroid cancer. Proceedings of the EORTCNCI-AACR Symposium on Molecular Targets and Cancer Therapeutics; 2008 Oct. 21-24; Geneva, Switzerland. Poster 379.
Kwak E L, Bang Y J, Camidge D R, Shaw A T, Solomon B, Maki R G, Ou S H, Dezube B J, Janne P A, Costa D B, Varella-Garcia M, Kim W H, Lynch T J, Fidias P, Stubbs H, Engleman J A, Sequist L V, Tan W, Gandhi L, Mino-Kenudson M, Wei G C, Shreeve S M, Ratain M J, Settleman J, Christensen J G, Haber D A, Wilner K, Slagia R, Shapiro G I, Clark J W, Iafrate A J. Anaplastic lymphoma kinase inhibition in non-small-cell lung cancer. N. Engl. J. Med. 363, 1693-1703 (2010).
Lam E T, Ringel M D, Kloos R T, et al. Phase II clinical trial of sorafenib in metastatic medullary thyroid cancer. J Clin Oncol 2010; 28:2323-30.
Lipson D, Capelletti M, Yelensky R, et al.: Identification of new ALK and RET gene fusions from colorectal and lung cancer biopsies. Nat Med 18:382-384, 2012
Liu S, Gong J, Morishita A, et al.: Use of protein array technology to investigate receptor tyrosine kinases activated in hepatocellular carcinoma. Exp Ther Med 2:399-403, 2011
Machens A, Niccoli-Sire P, Hoegel J, et al.: Early malignant progression of hereditary medullary thyroid cancer. N Engl J Med 349:1517-1525, 2003
Murakami H, Iwashita T, Asai N, et al.: Rho-dependent and -independent tyrosine phosphorylation of focal adhesion kinase, paxillin and p130Cas mediated by Ret kinase. Oncogene 18:1975-1982, 1999
Ohshima Y, Yajima I, Takeda K, Iida M, Kumasaka M, Matsumoto Y, Kato M. c-RET molecule in malignant melanoma from oncogenic RET-carrying transgenic mice and human cell lines. PLoS One. 2010 Apr. 21; 5(4):e10279.
Okayama H, Kohno T, Ishii Y, Shimada Y, Shiraishi K, Iwakawa R, Furuta K, Tsuta K, Shibata T, Yamamoto S, Watanabe S I, Sakamoto H, Kumamoto K, Takenoshita S, Gotoh N, Mizuno H, Sarai A, Kawano S, Yamaguchi R, Miyano S, Yokota J. Identification of Genes Upregulated in ALK-Positive and EGFR/KRAS/ALK-Negative Lung Adenocarcinomas. Cancer Res. 2012 Jan. 1; 72(1):100-111. Epub 2011 Nov. 11.
Pachnis V, Mankoo B, Costantini F. Expression of the c-ret proto-oncogene during mouse embryogenesis. Development. 1993 December; 119(4): 1005-17.
Panta G R, Nwariaku F, Kim L T: RET signals through focal adhesion kinase in medullary thyroid cancer cells. Surgery 136:1212-1217, 2004
Pao W, Girard N. New driver mutations in non-small-cell lung cancer. Lancet Oncol. 2011 February; 12(2):175-80. Review
Phay J E, Shah M H. Targeting RET receptor tyrosine kinase activation in cancer. Clin Cancer Res. 2010 Dec. 15; 16(24):5936-41. Epub 2010 Oct. 7. Review.
Plaza-Menacho I, Mologni L, Sala E, et al.: Sorafenib functions to potently suppress RET tyrosine kinase activity by direct enzymatic inhibition and promoting RET lysosomal degradation independent of proteasomal targeting. J Biol Chem 282:29230-29240, 2007
Ruhe J E, Streit S, Hart S, et al.: Genetic alterations in the tyrosine kinase transcriptome of human cancer cell lines. Cancer Res 67:11368-11376, 2007
Saiselet M, Floor S, Tarabichi M, et al.: Thyroid cancer cell lines: an overview. Front Endocrinol (Lausanne) 3:133, 2012
Schlumberger M J, Elisei R, Bastholt L, et al. Phase II study of safety and efficacy of motesanib in patients with progressive or symptomatic, advanced or metastatic medullary thyroid cancer. J Clin Oncol 2009; 27:3794-801.
Schuchardt A, D'Agati V, Larsson-Blomberg L, Costantini F, Pachnis V. Defects in the kidney and enteric nervous system of mice lacking the tyrosine kinase receptor Ret. Nature. 1994 Jan. 27; 367(6461):380-3.
Subramanian J, Velcheti V, Gao F, Govindan R. Presentation and stage-specific outcomes of lifelong never-smokers with non-small cell lung cancer (NSCLC). J Thorac Oncol. 2007 September; 2(9):827-30.
Takeuchi K, Soda M, Togashi Y, et al.: RET, ROS1 and ALK fusions in lung cancer. Nat Med 18:378-381, 2012
Tsuzuki T, Takahashi M, Asai N, Iwashita T, Matsuyama M, Asai J. Spatial and temporal expression of the ret proto-oncogene product in embryonic, infant and adult rat tissues. Oncogene. 1995 Jan. 5; 10(1):191-8.
Umezawa K. Inhibition of tumor growth by NF-kappaB inhibitors. Cancer Sci. 2006 October; 97(10):990-5. Epub 2006 Aug. 22. Review.
W. Obara, T. Tsunoda, K. Yoshida, M. Kanehira, R. Takata, T. Katagiri, T. Miki, T. Shuin, Y. Nakamura and T. Fujioka. Phase I/II study of novel HLA-A24 restricted DEPDC1 and MPHOSPH1 peptide vaccine for bladder cancer. Journal of Clinical Oncology, 2010 ASCO Annual Meeting Proceedings (Post-Meeting Edition). Vol 28, No 15_suppl (May 20 Supplement), 2010: e13122© 2010 American Society of Clinical Oncology.
Wang R, Hu H, Pan Y, et al.: RET Fusions Define a Unique Molecular and Clinicopathologic Subtype of Non-Small-Cell Lung Cancer. J Clin Oncol 30:4352-4359, 2012
Wells S A Jr, Gosnell J E, Gagel R F, et al. Vandetanib for the treatment of patients with locally advanced or metastatic hereditary medullarythyroid cancer. J Clin Oncol 2010; 28:767-72.
Zeng Q, Cheng Y, Zhu Q, Yu Z, Wu X, Huang K, Zhou M, Han S, Zhang Q. The relationship between over-expression of glial cell-derived neurotrophic factor and its RET receptor with progression and prognosis of human pancreatic cancer. J Int Med Res. 2008 July-August; 36(4):656-64.

### SEQUENCES

### SEQ ID NO: 1 RET

CCGAAGCAGGGCGCGCAGCAGCGCTGAGTGCCCCGGAACGTGCGTCGCGCCCCCAGTGTCCGTC
GCGTCCGCCGCGCCCCGGGCGGGGATGGGGCGGCCAGACTGAGCGCCGCACCCGCCATCCAGAC
CCGCCGGCCCTAGCCGCAGTCCCTCCAGCCGTGGCCCCAGCGCGCACGGGCGATGGCGAAGGCG
ACGTCCGGTGCCGCGGGGCTGCGTCTGCTGTTGCTGCTGCTGCTGCCGCTGCTAGGCAAAGTGG
CATTGGGCCTCTACTTCTCGAGGGATGCTTACTGGGAGAAGCTGTATGTGGACCAGGCGGCCGG
CACGCCCTTGCTGTACGTCCATGCCCTGCGGGACGCCCCTGAGGAGGTGCCCAGCTTCCGCCTG
GGCCAGCATCTCTACGGCACGTACCGCACACGGCTGCATGAGAACAACTGGATCTGCATCCAGG
AGGACACCGGCCTCCTCTACCTTAACCGGAGCCTGGACCATAGCTCCTGGGAGAAGCTCAGTGT
CCGCAACCGCGGCTTTCCCCTGCTCACCGTCTACCTCAAGGTCTTCCTGTCACCCACATCCCTT
CGTGAGGGCGAGTGCCAGTGGCCAGGCTGTGCCCGCGTATACTTCTCCTTCTTCAACACCTCCT
TTCCAGCCTGCAGCTCCCTCAAGCCCCGGGAGCTCTGCTTCCCAGAGACAAGGCCCTCCTTCCG
CATTCGGGAGAACCGACCCCCAGGCACCTTCCACCAGTTCCGCCTGCTGCCTGTGCAGTTCTTG
TGCCCCAACATCAGCGTGGCCTACAGGCTCCTGGAGGGTGAGGGTCTGCCCTTCCGCTGCGCCC
CGGACAGCCTGGAGGTGAGCACGCGCTGGGCCCTGGACCGCGAGCAGCGGGAGAAGTACGAGCT
GGTGGCCGTGTGCACCGTGCACGCCGGCGCGCGCGAGGAGGTGGTGATGGTGCCCTTCCCGGTG
ACCGTGTACGACGAGGACGACTCGGCGCCCACCTTCCCCGCGGGCGTCGACACCGCCAGCGCCG
TGGTGGAGTTCAAGCGGAAGGAGGACACCGTGGTGGCCACGCTGCGTGTCTTCGATGCAGACGT
GGTACCTGCATCAGGGGAGCTGGTGAGGCGGTACACAAGCACGCTGCTCCCCGGGGACACCTGG
GCCCAGCAGACCTTCCGGGTGGAACACTGGCCCAACGAGACCTCGGTCCAGGCCAACGGCAGCT
TCGTGCGGGCGACCGTACATGACTATAGGCTGGTTCTCAACCGGAACCTCTCCATCTCGGAGAA
CCGCACCATGCAGCTGGCGGTGCTGGTCAATGACTCAGACTTCCAGGGCCCAGGAGCGGGCGTC
CTCTTGCTCCACTTCAACGTGTCGGTGCTGCCGGTCAGCCTGCACCTGCCCAGTACCTACTCCC
TCTCCGTGAGCAGGAGGGCTCGCCGATTTGCCCAGATCGGGAAAGTCTGTGTGGAAAACTGCCA
GGCATTCAGTGGCATCAACGTCCAGTACAAGCTGCATTCCTCTGGTGCCAACTGCAGCACGCTA
GGGGTGGTCACCTCAGCCGAGGACACCTCGGGGATCCTGTTTGTGAATGACACCAAGGCCCTGC
GGCGGCCCAAGTGTGCCGAACTTCACTACATGGTGGTGGCCACCGACCAGCAGACCTCTAGGCA
GGCCCAGGCCCAGCTGCTTGTAACAGTGGAGGGGTCATATGTGGCCGAGGAGGCGGGCTGCCCC
CTGTCCTGTGCAGTCAGCAAGAGACGGCTGGAGTGTGAGGAGTGTGGCGGCCTGGGCTCCCCAA
CAGGCAGGTGTGAGTGGAGGCAAGGAGATGGCAAAGGGATCACCAGGAACTTCTCCACCTGCTC
TCCCAGCACCAAGACCTGCCCCGACGGCCACTGCGATGTTGTGGAGACCCAAGACATCAACATT
TGCCCTCAGGACTGCCTCCGGGGCAGCATTGTTGGGGGACACGAGCCTGGGGAGCCCCGGGGGA
TTAAAGCTGGCTATGGCACCTGCAACTGCTTCCCTGAGGAGGAGAAGTGCTTCTGCGAGCCCGA
AGACATCCAGGATCCACTGTGCGACGAGCTGTGCCGCACGGTGATCGCAGCCGCTGTCCTCTTC
TCCTTCATCGTCTCGGTGCTGCTGTCTGCCTTCTGCATCCACTGCTACCACAAGTTTGCCCACA
AGCCACCCATCTCCTCAGCTGAGATGACCTTCCGGAGGCCCGCCCAGGCCTTCCCGGTCAGCTA
CTCCTCTTCCAGTGCCCGCCGGCCCTCGCTGGACTCCATGGAGAACCAGGTCTCCGTGGATGCC
TTCAAGATCCTGGAGGATCCAAAGTGGGAATTCCCTCGGAAGAACTTGGTTCTTGGAAAAACTC
TAGGAGAAGGCGAATTTGGAAAAGTGGTCAAGGCAACGGCCTTCCATCTGAAAGGCAGAGCAGG
GTACACCACGGTGGCCGTGAAGATGCTGAAAGAGAACGCCTCCCCGAGTGAGCTTCGAGACCTG
CTGTCAGAGTTCAACGTCCTGAAGCAGGTCAACCACCCACATGTCATCAAATTGTATGGGGCCT
GCAGCCAGGATGGCCCGCTCCTCCTCATCGTGGAGTACGCCAAATACGGCTCCCTGCGGGGCTT
CCTCCGCGAGAGCCGCAAAGTGGGGCCTGGCTACCTGGGCAGTGGAGGCAGCCGCAACTCCAGC
TCCCTGGACCACCCGGATGAGCGGGCCCTCACCATGGGCGACCTCATCTCATTTGCCTGGCAGA
TCTCACAGGGGATGCAGTATCTGGCCGAGATGAAGCTCGTTCATCGGGACTTGGCAGCCAGAAA
CATCCTGGTAGCTGAGGGGCGGAAGATGAAGATTTCGGATTTCGGCTTGTCCCGAGATGTTTAT
GAAGAGGATTCGTACGTGAAGAGGAGCCAGGGTCGGATTCCAGTTAAATGGATGGCAATTGAAT
CCCTTTTTGATCATATCTACACCACGCAAAGTGATGTATGGTCTTTTGGTGTCCTGCTGTGGGA
GATCGTGACCCTAGGGGGAAACCCCTATCCTGGGATTCCTCCTGAGCGGCTCTTCAACCTTCTG
AAGACCGGCCACCGGATGGAGAGGCCAGACAACTGCAGCGAGGAGATGTACTGCCTGATGCTGC
AATGCTGGAAGCAGGAGCCGGACAAAAGGCCGGTGTTTGCGGACATCAGCAAAGACCTGGAGAA
GATGATGGTTAAGAGGAGAGACTACTTGGACCTTGCGGCGTCCACTCCATCTGACTCCCTGATT
TATGACGACGGCCTCTCAGAGGAGGAGACACCGCTGGTGGACTGTAATAATGCCCCCCTCCCTC
GAGCCCTCCCTTCCACATGGATTGAAAACAAACTCTATGGTAGAATTTCCCATGCATTTACTAG
ATTCTAGCACCGCTGTCCCCTCTGCACTATCCTTCCTCTCTGTGATGCTTTTTAAAAATGTTTC
TGGTCTGAACAAAAA

### SEQ ID NO: 2 DEPDC1

GACGCCACCGGGCGCTGACAGACCTATGGAGAGTCAGGGTGTGCCTCCCGGGCCTTATCGGGCC
ACCAAGCTGTGGAATGAAGTTACCACATCTTTTCGAGCAGGAATGCCTCTAAGAAAACACAGAC
AACACTTTAAAAAATATGGCAATTGTTTCACAGCAGGAGAAGCAGTGGATTGGCTTTATGACCT
ATTAAGAAATAATAGCAATTTTGGTCCTGAAGTTACAAGGCAACAGACTATCCAACTGTTGAGG
AAATTTCTTAAGAATCATGTAATTGAAGATATCAAAGGGAGGTGGGGATCAGAAAATGTTGATG
ATAACAACCAGCTCTTCAGATTTCCTGCAACTTCGCCACTTAAAACTCTACCACGAAGGTATCC
AGAATTGAGAAAAAACAACATAGAGAACTTTTCCAAAGATAAAGATAGCATTTTTAAATTACGA
AACTTATCTCGTAGAACTCCTAAAAGGCATGGATTACATTTATCTCAGGAAAATGGCGAGAAAA
TAAAGCATGAAATAATCAATGAAGATCAAGAAAATGCAATTGATAATAGAGAACTAAGCCAGGA
AGATGTTGAAGAAGTTTGGAGATATGTTATTCTGATCTACCTGCAAACCATTTTAGGTGTGCCA
TCCCTAGAAGAAGTCATAAATCCAAAACAAGTAATTCCCCAATATATAATGTACAACATGGCCA
ATACAAGTAAACGTGGAGTAGTTATACTACAAAACAAATCAGATGACCTCCCTCACTGGGTATT
ATCTGCCATGAAGTGCCTAGCAAATTGGCCAAGAAGCAATGATATGAATAATCCAACTTATGTT
GGATTTGAACGAGATGTATTCAGAACAATCGCAGATTATTTTCTAGATCTCCCTGAACCTCTAC
TTACTTTTGAATATTACGAATTATTTGTAAACATTTTGGGCTTGCTGCAACCTCATTTAGAGAG
GGTTGCCATCGATGCTCTACAGTTATGTTGTTTGTTACTTCCCCCACCAAATCGTAGAAAGCTT
CAACTTTTAATGCGTATGATTTCCCGAATGAGTCAAAATGTTGATATGCCCAAACTTCATGATG
CAATGGGTACGAGGTCACTGATGATACATACCTTTTCTCGATGTGTGTTATGCTGTGCTGAAGA
AGTGGATCTTGATGAGCTTCTTGCTGGAAGATTAGTTTCTTTCTTAATGGATCATCATCAGGAA
ATTCTTCAAGTACCCTCTTACTTACAGACTGCAGTGGAAAAACATCTTGACTACTTAAAAAAGG
GACATATTGAAAATCCTGGAGATGGACTATTTGCTCCTTTGCCAACTTACTCATACTGTAAGCA
GATTAGTGCTCAGGAGTTTGATGAGCAAAAAGTTTCTACCTCTCAAGCTGCAATTGCAGAACTT
TTAGAAAATATTATTAAAAACAGGAGTTTACCTCTAAAGGAGAAAAGAAAAAAACTAAAACAGT
TTCAGAAGGAATATCCTTTGATATATCAGAAAAGATTTCCAACCACGGAGAGTGAAGCAGCACT
TTTTGGTGACAAACCTACAATCAAGCAACCAATGCTGATTTTAAGAAAACCAAAGTTCCGTAGT
CTAAGATAACTAACTGAATTAAAAATTATGTAATACTTGTGGAACTTTGATAAATGAAGCCATA
TCTGAGAATGTAGCTACTCAAAAGGAAGTCTGTCATTAATAAGGTATTTCTAAATAAACACATT
ATGTAAGGAAGTGCCAAAATAGTTATCAATGTGAGACTCTTAGGAAACTAACTAGATCTCAATT
GAGAGCACATAACAATAGATGATACCAAATACTTTTTGTTTTTAACACAGCTATCCAGTAAGGC
TATCATGATGTGTGCTAAAATTTTATTTACTTGAATTTTGAAAACTGAGCTGTGTTAGGGATTA
AACTATAATTCTGTTCTTAAAAGAAAATTTATCTGCAAATGTGCAAGTTCTGAGATATTAGCTA
ATGAATTAGTTGTTTGGGGTTACTTCTTTGTTTCTAAGTATAAGAATGTGAAGAATATTTGAAA
ACTCAATGAAATAATTCTCAGCTGCCAAATGTTGCACTCTTTTATATATTCTTTTTCCACTTTT
GATCTATTTATATATATGTATGTGTTTTTAAAATATGTGTATATTTTATCAGATTTGGTTTTGC
CTTAAATATTATCCCCAATTGCTTCAGTCATTCATTTGTTCAGTATATATATTTTGAATTCTAG
TTTTCATAATCTATTAGAAGATGGGGATATAAAAGAAGTATAAGGCAATCATATATTCATTCAA
AAGATATTTATTTAGCAACTGCTATGTGCCTTTCGTTGTTCCAGATATGCAGAGACAATGATAA
ATAAAACATATAATCTCTTCCATAAGGTATTTATTTTTTAATCAAGGGAGATACACCTATCAGA
TGTTTAAAATAACAACACTACCCACTGAAATCAGGGCATATAGAATCATTCAGCTAAAGAGTGA
CTTCTATGATGATGGAACAGGTCTCTAAGCTAGTGGTTTTCAAACTGGTACACATTAGACTCAC
CCGAGGAATTTTAAAACAGCCTATATGCCCAGGGCCTAACTTACACTAATTAAATCTGAATTTT
GGGGATGTTGTATAGGGATTAGTATTTTTTTTAATCTAGGTGATTCCAATATTCAGCCAACTGT
GAGAATCAATGGCCTAAATGCTTTTTATAAACATTTTTATAAGTGTCAAGATAATGGCACATTG
ACTTTATTTTTTCATTGGAAGAAAATGCCTGCCAAGTATAAATGACTCTCATCTTAAAACAAGG
TTCTTCAGGTTTCTGCTTGATTGACTTGGTACAAACTTGAAGCAAGTTGCCTTCTAATTTTTAC
TCCAAGATTGTTTCATATCTATTCCTTAAGTGTAAAGAAATATATAATGCATGGTTTGTAATAA
AATCTTAATGTTTAATGACTGTTCTCATTTCTCAATGTAATTTCATACTGTTTCTCTATAAAAT
GATAGTATTCCATTTAACATTACTGATTTTTATTAAAAACCTGGACAGAAAATTATAAATTATA
AATATGACTTTATCCTGGCTATAAAATTATTGAACCAAAATGAATTCTTTCTAAGGCATTTGAA
TACTAAAACGTTTATTGTTTATAGATATGTAAAATGTGGATTATGTTGCAAATTGAGATTAAAA
TTATTTGGGGTTTTGTAACAATATAATTTTGCTTTTGTATTATAGACAAATATATAAATAATAA
AGGCAGGCAACTTTCATTTGCAAAAAAAAAAAAAA

### SEQ ID NO: 12 KIF5B

CCGAAGCAGGGCGCGCAGCAGCGCTGAGTGCCCCGGAACGTGCGTCGCGCCCCCAGTGTCCGTC
GCGTCCGCCGCGCCCCGGGCGGGGATGGGGCGGCCAGACTGAGCGCCGCACCCGCCATCCAGAC
CCGCCGGCCCTAGCCGCAGTCCCTCCAGCCGTGGCCCCAGCGCGCACGGGCGATGGCGAAGGCG
ACGTCCGGTGCCGCGGGGCTGCGTCTGCTGTTGCTGCTGCTGCTGCCGCTGCTAGGCAAAGTGG
CATTGGGCCTCTACTTCTCGAGGGATGCTTACTGGGAGAAGCTGTATGTGGACCAGGCGGCCGG
CACGCCCTTGCTGTACGTCCATGCCCTGCGGGACGCCCCTGAGGAGGTGCCCAGCTTCCGCCTG
GGCCAGCATCTCTACGGCACGTACCGCACACGGCTGCATGAGAACAACTGGATCTGCATCCAGG
AGGACACCGGCCTCCTCTACCTTAACCGGAGCCTGGACCATAGCTCCTGGGAGAAGCTCAGTGT
CCGCAACCGCGGCTTTCCCCTGCTCACCGTCTACCTCAAGGTCTTCCTGTCACCCACATCCCTT
CGTGAGGGCGAGTGCCAGTGGCCAGGCTGTGCCCGCGTATACTTCTCCTTCTTCAACACCTCCT
TTCCAGCCTGCAGCTCCCTCAAGCCCCGGGAGCTCTGCTTCCCAGAGACAAGGCCCTCCTTCCG
CATTCGGGAGAACCGACCCCCAGGCACCTTCCACCAGTTCCGCCTGCTGCCTGTGCAGTTCTTG
TGCCCCAACATCAGCGTGGCCTACAGGCTCCTGGAGGGTGAGGGTCTGCCCTTCCGCTGCGCCC
CGGACAGCCTGGAGGTGAGCACGCGCTGGGCCCTGGACCGCGAGCAGCGGGAGAAGTACGAGCT
GGTGGCCGTGTGCACCGTGCACGCCGGCGCGCGCGAGGAGGTGGTGATGGTGCCCTTCCCGGTG
ACCGTGTACGACGAGGACGACTCGGCGCCCACCTTCCCCGCGGGCGTCGACACCGCCAGCGCCG
TGGTGGAGTTCAAGCGGAAGGAGGACACCGTGGTGGCCACGCTGCGTGTCTTCGATGCAGACGT
GGTACCTGCATCAGGGGAGCTGGTGAGGCGGTACACAAGCACGCTGCTCCCCGGGGACACCTGG
GCCCAGCAGACCTTCCGGGTGGAACACTGGCCCAACGAGACCTCGGTCCAGGCCAACGGCAGCT
TCGTGCGGGCGACCGTACATGACTATAGGCTGGTTCTCAACCGGAACCTCTCCATCTCGGAGAA
CCGCACCATGCAGCTGGCGGTGCTGGTCAATGACTCAGACTTCCAGGGCCCAGGAGCGGGCGTC
CTCTTGCTCCACTTCAACGTGTCGGTGCTGCCGGTCAGCCTGCACCTGCCCAGTACCTACTCCC
TCTCCGTGAGCAGGAGGGCTCGCCGATTTGCCCAGATCGGGAAAGTCTGTGTGGAAAACTGCCA
GGCATTCAGTGGCATCAACGTCCAGTACAAGCTGCATTCCTCTGGTGCCAACTGCAGCACGCTA
GGGGTGGTCACCTCAGCCGAGGACACCTCGGGGATCCTGTTTGTGAATGACACCAAGGCCCTGC
GGCGGCCCAAGTGTGCCGAACTTCACTACATGGTGGTGGCCACCGACCAGCAGACCTCTAGGCA
GGCCCAGGCCCAGCTGCTTGTAACAGTGGAGGGGTCATATGTGGCCGAGGAGGCGGGCTGCCCC
CTGTCCTGTGCAGTCAGCAAGAGACGGCTGGAGTGTGAGGAGTGTGGCGGCCTGGGCTCCCCAA
CAGGCAGGTGTGAGTGGAGGCAAGGAGATGGCAAAGGGATCACCAGGAACTTCTCCACCTGCTC
TCCCAGCACCAAGACCTGCCCCGACGGCCACTGCGATGTTGTGGAGACCCAAGACATCAACATT
TGCCCTCAGGACTGCCTCCGGGGCAGCATTGTTGGGGGACACGAGCCTGGGGAGCCCCGGGGGA
TTAAAGCTGGCTATGGCACCTGCAACTGCTTCCCTGAGGAGGAGAAGTGCTTCTGCGAGCCCGA
AGACATCCAGGATCCACTGTGCGACGAGCTGTGCCGCACGGTGATCGCAGCCGCTGTCCTCTTC
TCCTTCATCGTCTCGGTGCTGCTGTCTGCCTTCTGCATCCACTGCTACCACAAGTTTGCCCACA
AGCCACCCATCTCCTCAGCTGAGATGACCTTCCGGAGGCCCGCCCAGGCCTTCCCGGTCAGCTA
CTCCTCTTCCAGTGCCCGCCGGCCCTCGCTGGACTCCATGGAGAACCAGGTCTCCGTGGATGCC
TTCAAGATCCTGGAGGATCCAAAGTGGGAATTCCCTCGGAAGAACTTGGTTCTTGGAAAAACTC
TAGGAGAAGGCGAATTTGGAAAAGTGGTCAAGGCAACGGCCTTCCATCTGAAAGGCAGAGCAGG
GTACACCACGGTGGCCGTGAAGATGCTGAAAGAGAACGCCTCCCCGAGTGAGCTTCGAGACCTG
CTGTCAGAGTTCAACGTCCTGAAGCAGGTCAACCACCCACATGTCATCAAATTGTATGGGGCCT
GCAGCCAGGATGGCCCGCTCCTCCTCATCGTGGAGTACGCCAAATACGGCTCCCTGCGGGGCTT
CCTCCGCGAGAGCCGCAAAGTGGGGCCTGGCTACCTGGGCAGTGGAGGCAGCCGCAACTCCAGC
TCCCTGGACCACCCGGATGAGCGGGCCCTCACCATGGGCGACCTCATCTCATTTGCCTGGCAGA
TCTCACAGGGGATGCAGTATCTGGCCGAGATGAAGCTCGTTCATCGGGACTTGGCAGCCAGAAA
CATCCTGGTAGCTGAGGGGCGGAAGATGAAGATTTCGGATTTCGGCTTGTCCCGAGATGTTTAT
GAAGAGGATTCGTACGTGAAGAGGAGCCAGGGTCGGATTCCAGTTAAATGGATGGCAATTGAAT
CCCTTTTTGATCATATCTACACCACGCAAAGTGATGTATGGTCTTTTGGTGTCCTGCTGTGGGA
GATCGTGACCCTAGGGGGAAACCCCTATCCTGGGATTCCTCCTGAGCGGCTCTTCAACCTTCTG
AAGACCGGCCACCGGATGGAGAGGCCAGACAACTGCAGCGAGGAGATGTACTGCCTGATGCTGC
AATGCTGGAAGCAGGAGCCGGACAAAAGGCCGGTGTTTGCGGACATCAGCAAAGACCTGGAGAA
GATGATGGTTAAGAGGAGAGACTACTTGGACCTTGCGGCGTCCACTCCATCTGACTCCCTGATT
TATGACGACGGCCTCTCAGAGGAGGAGACACCGCTGGTGGACTGTAATAATGCCCCCCTCCCTC
GAGCCCTCCCTTCCACATGGATTGAAAACAAACTCTATGGTAGAATTTCCCATGCATTTACTAG
ATTCTAGCACCGCTGTCCCCTCTGCACTATCCTTCCTCTCTGTGATGCTTTTTAAAAATGTTTC
TGGTCTGAACAAAAA

### SEQ ID NO: 13 Coiled Coil Domain Containing 6

AGTGCAATACTGCCCAAGCCCGGGCGGGGTCTCTGTTCTCTGGCAGAGGAGGTCCCTTGGCAGC
GGGAAGCGCCCTCTCTTTCTCTCGCCGCCGCTCCGAGTCTGCGCCCTGGTGCCAGGCGCTCAGC
TCGGCGCTCCCCTGTGCTCGCCCGGCGCCCACTCATTCGCAGCCCGGCCTTCGTCGCCGCCGCC
TCCCTGCTGCTCCTCCTCCTTTCCCCAGCCCGCCGCGGCCATGGCGGACAGCGCCAGCGAGAGC
GACACGGACGGGGCGGGGGGCAACAGCAGCAGCTCGGCCGCCATGCAGTCGTCCTGCTCGTCGA
CCTCGGGCGGCGGCGGTGGCGGCGGGGGAGGCGGCGGCGGTGGGAAGTCGGGGGGCATTGTCAT
CTCGCCGTTCCGCCTGGAGGAGCTCACCAACCGCCTGGCCTCGCTGCAGCAAGAGAACAAGGTG
CTGAAGATAGAGCTGGAGACCTACAAACTGAAGTGCAAGGCACTGCAGGAGGAGAACCGCGACC
TGCGCAAAGCCAGCGTGACCATCCAAGCCAGGGCTGAGCAGGAAGAAGAATTCATTAGTAACAC
TTTATTCAAGAAAATTCAGGCTTTGCAGAAGGAGAAAGAAACCCTTGCTGTAAATTATGAGAAA
GAAGAAGAATTCCTCACTAATGAGCTCTCCAGAAAATTGATGCAGTTGCAGCATGAGAAAGCCG
AACTAGAACAGCATCTTGAACAAGAGCAGGAATTTCAGGTCAACAAACTGATGAAGAAAATTAA
AAAACTGGAGAATGACACCATTTCTAAGCAACTTACATTAGAACAGTTGAGACGGGAGAAGATT
GACCTTGAAAATACATTGGAACAAGAACAAGAAGCACTAGTTAATCGCCTCTGGAAAAGGATGG
ATAAGCTTGAAGCTGAAAAGCGAATCCTGCAGGAAAAATTAGACCAGCCCGTCTCTGCTCCACC
ATCGCCTAGAGATATCTCCATGGAGATTGATTCTCCAGAAAATATGATGCGTCACATCAGGTTT
TTAAAGAATGAAGTGGAACGGCTGAAGAAGCAACTGAGAGCTGCTCAGTTACAGCATTCAGAGA
AAATGGCACAGTATCTGGAGGAGGAACGTCACATGAGAGAAGAGAACTTGAGGCTCCAGAGGAA
GCTGCAGAGGGAGATGGAGAGAAGAGAAGCCCTCTGTCGACAGCTCTCCGAGAGTGAGTCCAGC
TTAGAAATGGACGACGAAAGGTATTTTAATGAGATGTCTGCACAAGGATTAAGACCTCGCACTG
TGTCCAGCCCGATCCCTTACACACCTTCTCCGAGTTCAAGCAGGCCTATATCACCTGGTCTATC
ATATGCAAGTCACACGGTTGGTTTCACGCCACCAACTTCACTGACTAGAGCTGGAATGTCTTAT
TACAATTCCCCGGGTCTTCACGTGCAGCACATGGGAACATCCCATGGTATCACAAGGCCTTCAC
CACGGAGAAGCAACAGTCCTGACAAATTCAAACGGCCCACGCCGCCTCCATCTCCCAACACACA
GACCCCAGTCCAGCCACCTCCGCCTCCACCTCCGCCACCCATGCAGCCCACGGTCCCCTCAGCA
GCCACCTCGCAGCCTACTCCTTCGCAACATTCGGCGCACCCCTCCTCCCAGCCTTAATGCATGA
GCTTAGTCTGAATTTCAAGTTGGGACTCATCCAATGGAGCCGTCTACTCAACGCCAAAGGCTTC
CTTCTCTGGCATATTTGGATATGACTTATTTGCACTGAGGTTATCTAGGCTTCACTATCCATTG
TGTTGTAAATGTTTGTCAGAAATGCAGCCAGTGTTGTGGGTCTACAACACTAACCAGACGACTT
TTTCCATCAGTGTTTTACTTGAATCTTCATGTACGTCCATTCCCTGGCTGGAACCTTCGCTGTT
TGGTATTTGGTATTTCAGCAGCAGTGTGCAATTTTTGCTTGGCCCAGAGCTTCATTCTCCTGGC
TTTTAGGTTTGTAAAAGAAAAAGGGATATCTTTTTTATATTTTTTTCCATGAATCTGCAGAAAA
TTACTGAGCTGTTGTTACCCTCCTCTCATTATAATAGTGTTTACCAAACATACCAATAATTCAG
CACTACAATTCAGACCTTTGAAAATCTGGCTTTCAGTGTAGAACAGAAAGTTAGATGAATCAGT
GCCCAAGACATATTTTCTGTTTAACAGAACTTTCTACAGATACATTTTTTACAGGTTATTTTCA
TTGTGTTATTGACATCCATGTCTCTCGTAAAACAGATGGCCCAAAGTAATGAATCATGTGGCTG
TACCTTCTCCACATAAATGGGATGGATAATTATCGTATATTAAGATGTGATTCTCTTTTTTATC
CTTAATGTTAATCTACTTAACCTGGCCCCCTCTAACATGAGTCGATAAATGTTGTCCTACTCAC
CGGTGGTTTCAATGGCTAATTAGAATGTGTTATTTGATTTCTGCTGCAGAAGGCAGTGTGATTG
TAACAAAAACAATGCGGCTTCCCCCTTTCGTACTTCATTTGTGTTCTCTTAAAATAGAGTTTGA
ACAAATATTTTAAAGGTGCAAAATACCATTAGAAAATACTATTTGAAATGGACATTATCGCATT
ATCTTGGCATAATGGCCAGAAAATATTGTATTGCTTGGCAGAAAAGAAAATAAGGTCTAAAGGA
AAGTAGCACATTAGCATTGATGGCTGTTCATTTCACCCAGTATAAGCAAGTGCAGTGTACAAAG
AAGTATATTCTGAATACATTATTTCCATTCATTTAGCACAAATAAATCATTTGGTTTCACTTTG
CAGTGGAACACTGAGTCACTCTTTTCTTAACACGTGCAACATCTTAATTTTTGTTTTTCAGCAG
TTGCTGTTTTGTACTTTGGTAGTAAAGTGATTTTTACCACCTGTGTTTGCATATTTATATATGC
TGTGGATGAAAATAACTTACTAGAGAATGTATATTTTATGACAAGAATGTGTATCTGTTGGATA
TAATCAGAGAACTGAAAAGTAATTTATCAGTAATTTTTAAGAGTCCATGTTTTGTGACAACCAT
CTCTAATAGCCAACTCTTTATTAAACACACTCCTAAAAATAAGGAACCATGACATTGTAGATAT
TTAATATTGTACAGTATAGAAACCTCCATTTTTGCCTTCGAATGCATATTTAAGAGTTAACAGA
ATGAAAAAAAAAAGTCTTGTTGGATAATAGTGTTTGACTAGCGTTTTAAGAACTTGAGAGTAAA
AGCAACAATAAGATTTTTTCACCTCTTCCTGCTTCCACCCCCAAACTGAGAACATCACTCAATT
GTTTGGAAGAAACTGTAGGTCTATATAAATTTTATTTATAATGTATGTGTAATATACATAATCA
TAATACAGTTCTCAGATGCAGGGAAGAAGTTTGGCATTTAATCATTGAGGCTTTAGGTTTTTGA
TGTGATCAGACTGGGCCATGTCAAACCCGGAATTTTCACCAACAGTTCACTCACCCTCCTGGTA
CATTGCCATTCCAAGGAATTCTGAGAGTAGGCAAACAAATTTTGCCTTCATGGTACAGTTCTCA
GTTTTTCTTATAGGAGAAATATGGTATATGTTTATAAGAATCTTTTATGAGATTATAGATTTCA
ATGCTGTGGATAGTGTCTTGCACCCAAACAAGAAAGTCCATAATGGAATGATCTTCCCTCAGCT
TCCTATCGATTTAGTTACCTCTTGAAAGCACAAAAATTAAAACATTGCCATATGTTGAATTTTT
AAAAAGCACTTGGAGTGAGCGAACATTTCCTGATAAATGCCTTTTAGAGATAGGTTCTTGATAT
TCAGACATCTGCAGAAATGTTCTGGTTCCCAAAGTCATTTCACTTCGAAATAAAACACAGCTCC
TTCAAACAGCACTTTTTCCACATAAATCTAGTTGCCTCTCCCTGTGGACATTCAGAACTGATAG
AACAAACACTACTCTTTTGAATTTGATGGTTCGTGTCCTTTAAAGTGTTTGAGGACCTATGCAG
AGCCTGTAACACTTGGGTAGTACCTGCTAGGACAATTTCTTGGCAATTGTCTTACTACTAGGGA
TCAGTAAGATTTAGATTCTGAGCCCATAATGGCAACAGCCCCCTCACCTATGGGAAGCTGACTT
CCCTCAGTCGGGCACTTCTCATGGGGGCTGAACATGGTTCCTGCCATTCTGTTACCCACTCTCC
CAGGTGAGCCCTGGATTGGCTCCCAGAAGGCCTTTGTAAAATCAGTAGCCGTCCTGCAGGCAGG
TGGGAGCAACAGGGGCTTCAGTAGCTTCATTTTCCTGTCTTGCAGACAGAGACCCTTGGCTACC
ACTGTGCTGCTAATAGGATAAGTACTCTGTTGCCAGATTACCATGCCTTTTATACAAAACCAAA
TTAACTTACCTAATACCTGACACCTCTTTGGGCTCTGAACTGCTTTCTCTCATCAAGCATGCTA
GCACTCTAGACAGAATTCTAGAAATTTGGCAGATAGTGGAAGCCTTTAATTGAACTTACTCCTT
CGTTGACTGAAAGGAGTTTTAAATTCTGAGCTCCTGAGATACTGACTAGCAACCATGGAATGAA
TGTGTGACCAGAAAGTGGCTTTGACACCAAGTGCTACTGTCCCTTTGTAATTGGCTTCTAACAG
AATTCAACCAGAAATAATTGATAATGTGAATTTTTGTTAATTGTTCACTTGTAGGAAAATAGAA
CATGTATCACCCTTTGTTAGGTAGACATGAACTTTTCCTGCACAAAGCCTTGCTTTTAGAGAAT
GCCCAATAAGGCAAGAAAAAGCATAGTAACTTGTGCTTTGAGAGCTCAATATTTGTATCTTATC
AGTACAGAAGAAATATTTCTGTGTAACTTGATCTTCTGTCTAGTACTTGTCTTATAGGTAACCA
ACACTGAAAACTTTGTAGTGATGACTACCAAAGAAATACATAGTAAAACAACCTTTTATTTCCA
AATTGTTAAAGAGCCAGCCATTGATGCTGCTACATGAGTTCCATGCTCAAGAGCCATTGTAAGA
GATTAAGGGGTTTCTAGGTTTTTGGTGATTTTTTGTTTGTTTTTTTCTTTGTTTTTTAGGGTTT
TTTTTTCTTCTTTAATTTTTTGATTAAAACATACACACAGCTGTTAGCATAAAGTCGTGGGGGG
CATTTTCTGGAATGCTCAGCAGTTCTGATTAACTGCCAAGCCCAGGTTGCCTCTCATGAGGCAA
CTGAAAAAATCCTGTGTCTTGATAGCATGGGTGCTGTGTGTGTGCATGTGTGTGTCTGCATTCA
TGCCTTAACTCGGGTTACTGCACAACTTTAGTTCTTGACTTAGTCTGCACCGTCATCTAGATTG
TATTGTACATCTCGGTCTGAACTTCATCCTGGCAAAAACAAAGTTGCAGGCACAACAGTTTAAG
AATGCATTCCTCCAGAAGAGTATCTGGTCAGGTTGACCCCTGAGCCTTCTTTGGACTTGATTTG
GAACTTAGCCTGGAAAGCGAAAGTGGACTGTCCAACAGAAAGATGTCAACAAGGAAAAGAGGAG
AGCCAAGCGCTAGCATGCCTTTTGCCTCTGCATATCTGTGCACACTGTATGTTGTTCATGATAG
CTTGTCTACAACTTGACTAGGTTGGAGTTCTGGTAATAGTGGCAATCTTGACATTCTTGGTCAG
AGTTTAGAGAGATGTAAGACTTTCAATTAATGTCTTATTTACTCCTTTATGTTGATTAGTCTTT
GATACATGTGCTGAATCAGAAACCTAAATAAAGATAATTTTTTAAAATGTACCTCTTGAGCCTT
AAAAAAAAAAAAAAAAAA

What is claimed is:

1. A method of diagnosing a RET or DEPDC1 related cancer comprising detecting the presence of or measuring the amount of nucleic acid associated with a nucleic acid variation, over-expression, truncation, or fusions of DEP domain containing 1 gene (DEPDC1) or RET from a tissue sample from the subject; wherein an increase in the amount of amplification product or labeled probe relative to a control indicates the presence of RET or DEPDC1 related cancer.

2. The method of claim 1, wherein the nucleic acid is measured by measuring mRNA levels by conducting a first reverse transcription polymerase chain reaction (RT-PCR), real time polymerase chain reaction (PCR), or real-time RT-PCR on the sample.

3. The method of claim 2, wherein the RT-PCR, real-time PCR, or real-time RT-PCR reaction comprises the use of a reverse primer capable of specifically hybridizing to one or more DEPDC1 target sequences and at least one or more forward primers.

4. The method of claim 3, wherein the reverse primer comprises SEQ ID NO: 10, SEQ ID NO 19, SEQ ID NO: 22, or SEQ ID NO 25.

5. The method of claim 3, wherein the forward primer comprises SEQ ID NO: 9, SEQ ID NO 18, SEQ ID NO: 21, or SEQ ID NO: 24.

6. The method of claim 2, wherein the RT-PCR, real-time PCR, or real-time RT-PCR reaction comprises the use of a reverse primer capable of specifically hybridizing to one or more RET target sequences and at least one or more forward primers.

7. The method of claim 6, wherein at least one reverse primer comprises SEQ ID NO: 4, SEQ ID NO: 7, SEQ ID NO: 13, or SEQ ID NO: 16.

8. The method of claim 6 wherein the reverse and forward primers are specific for the kinase region of the target sequence.

9. The method of claim 8, wherein the reverse primer comprises SEQ ID NO: 4, SEQ ID NO: 13, or SEQ ID NO: 16.

10. The method of claim 8, wherein the forward primer comprises SEQ ID NO: 3, SEQ ID NO: 12, or SEQ ID NO: 15.

11. The method of claim 6, wherein the forward primer comprises SEQ ID NO: 3, SEQ ID NO: 6, SEQ ID NO: 12, or SEQ ID NO: 15.

12. The method of claim 6, further comprising the use of at least one forward primer that specifically hybridizes to a RET sequence 5′ of a fusion breakpoint.

13. The method of claim 12, wherein at least one forward primer comprises SEQ ID NO: 6.

14. The method of claim 12, wherein at least one reverse primer comprises SEQ ID NO: 7.

15. The method of claim 6, further comprising the use of at least one forward primer that specifically hybridizes to a kinesin-1 heavy chain gene (KIF5B) gene sequence 5′ of a fusion breakpoint; a coiled coil domain containing 6 (CCDC6) gene sequence 5′ of a fusion breakpoint; or a nuclear receptor coactivator 4 (NCOA4) gene sequence 5′ of a fusion breakpoint.

16. The method of claim 1, wherein the nucleic acid is measured by microarray or in-situ hybridization method.

17. The method of claim 16, wherein the method comprises using a probe which specifically hybridizes to RET or DEPDC1.

18. The method of claim 17, wherein the probe comprises SEQ ID NO: 5, SEQ ID NO 14, or SEQ ID NO: 17.

19. The method of claim 17, wherein the probe comprises SEQ ID NO: 11, SEQ ID NO: 20, SEQ ID NO: 23, or SEQ ID NO: 26.

20. The method of claim 17, further comprising the use of a second probe that specifically binds to RET 5′ of a fusion breakpoint.

21. The method of claim 20, wherein the second probe comprises SEQ ID NO: 8.

22. The method of claim 1, wherein the cancer is selected from the group consisting of neuroblastoma, breast cancer, ovarian cancer, colorectal carcinoma, non-small cell lung carcinoma (NSCLC), diffuse large B-cell lymphoma, esophageal squamous cell carcinoma, anaplastic large-cell lymphoma, neuroblastoma, inflammatory myofibroblastic tumors, malignant histiocytosis, and glioblastomas.

23. The method of claim 22, wherein the cancer is an EGFR, KRAS, ALK negative NSCLC.

24. A method of screening for an agent that inhibits a RET or DEPDC1 related cancer in a subject comprising
a) obtaining a tissue sample from a subject with an RET or DEPDC1 related cancer;
b) contacting the tissue sample with the agent
c) extracting mRNA from the tissue sample;
d) conducting an RT-PCR reaction on the mRNA from the tissue sample;
wherein the RT-PCR reaction comprises a reverse primer capable of specifically hybridizing to one or more RET or DEPDC1 sequences and at least one forward primer; and
wherein a decrease in the amount of amplification product relative to an untreated control indicates an agent that can inhibit an RET or DEPDC1 related cancer.

25. A kit for diagnosing an RET or DEPDC1 related cancer comprising (a) a first primer labeled with a first detection reagent, wherein said first primer is a reverse primer, wherein said reverse primer is one or more polynucleotide(s) that hybridizes to RET or DEPDC1 3′ to the kinase region of RET or to a region of DEPDC1; and (b) at least one second primer, wherein said second primer is a forward primer, wherein said forward primer is one or more polynucleotide(s) that hybridizes to RET or DEPDC1, or KIF5B.

26. The kit of claim 25, wherein the reverse primer is SEQ ID NO: 4, SEQ ID NO: 7, SEQ ID NO: 10, SEQ ID NO: 13, SEQ ID NO: 16, SEQ ID NO: 19, SEQ ID NO: 22, or SEQ ID NO: 25.

27. The kit of claim 25, wherein the at least one or more forward primer comprises at least one forward primer selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 21, and SEQ ID NO: 24.

28. The kit of claim 25, further comprising a forward primer that specifically hybridizes to wild-type RET 5′ of any potential fusion breakpoint.

29. The kit of claim 28, wherein the forward primer comprises SEQ ID NO: 6.

30. The kit of claim 25, further comprising a forward primer that specifically hybridizes to the RET kinase domain or DEPDC1.

31. The kit of claim 30, wherein the forward primer is selected from the group consisting of SEQ ID NO: 3, SEQ ID NO: 12, and SEQ ID NO: 15.

32. The kit of claim 30, wherein the forward primer is selected from the group consisting of SEQ ID NO: 9, SEQ ID NO: 18, SEQ ID NO: 21, and SEQ ID NO: 24.

33. The kit of claim 25, further comprising a control primer pair.

34. The kit of claim 25, wherein the first and second primers are labeled with a first and second detection reagent, respectively.

35. A method of diagnosing a RET or DEPDC1 related cancer in a subject comprising measuring the expression level of RET or DEPDC1 in a tissue sample form the subject, wherein increased expression of RET or DEPDC1 relative to a cancer free control indicates the presence of a RET or DEPDC1 related cancer.

36. A method of diagnosing a RET related cancer in a subject comprising conducting a nucleic acid amplification process on a tissue sample from the subject and detecting the presence of or measuring the amount of nucleic acid associated with RET kinase domain in the tissue sample, wherein the presence or an increase in RET kinase relative to a control indicates the presence of an RET related cancer.

37. The method of claim 36, wherein the nucleic acid amplification process comprises reverse transcription polymerase chain reaction (RT-PCR), real-time PCR, or real-time RT-PCR.

38. The method of claim 37, wherein the RT-PCR, real-time PCR, or real-time RT-PCR reaction comprises at least one reverse primer that specifically hybridizes to or 3′ to a RET kinase domain.

39. The method of claim 38, wherein at least one reverse primer comprises SEQ ID NO: 4, SEQ ID NO: 13 or SEQ ID NO: 16.

40. The method of claim 38, wherein the RT-PCR, real-time PCR, or real-time RT-PCR reaction further comprises at least one forward primer that specifically hybridizes to a RET kinase domain.

41. The method of claim 40, wherein at least one forward primer comprises SEQ ID NO: 3, SEQ ID NO: 12, or SEQ ID NO: 15.

42. The method of claim 36, where the method further comprises the use of a forward primer that specifically hybridizes to RET 5′ of a fusion breakpoint.

43. The method of claim 42, wherein at least one forward primer comprises SEQ ID NO: 6.

44. The method of claim 42, wherein the method further comprises the use of a reverse primer that specifically hybridizes to RET 5′ of a fusion breakpoint.

45. The method of claim 44, wherein at least one reverse primer comprises SEQ ID NO: 7.

46. The method of claim 36, wherein the cancer is selected from the group consisting of neuroblastoma, breast cancer, ovarian cancer, colorectal carcinoma, non-small cell lung carcinoma (NSCLC), diffuse large B-cell lymphoma, esophageal squamous cell carcinoma, anaplastic large-cell lymphoma, neuroblastoma, inflammatory myofibroblastic tumors, malignant histiocytosis, and glioblastomas.

47. The method of claim 46, wherein the cancer is a EGFR, KRAS, ALK negative NSCLC.
